SEQUENCE LISTING (38 sequences)

SEQ ID NO: 1 - DNA, 1932 bp, Bacillus thuringiensis
  CDS (1)..(1932): Native cry3A coding sequence according to Sekar et al. 1987, Proc. Natl. Aca. Sci. 847036-7040.
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatggatcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaatttctggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggttcatcttatgaatcttgggtaaactttaaccgttatcgcagagagatgacattaacagtattagatttaattgcactatttccattgtatgatgttcggctatacccaaaagaagttaaaaccgaattaacaagagacgttttaacagatccaattgtcggagtcaacaaccttaggggctatggaacaaccttctctaatatagaaaattatattcgaaaaccacatctatttgactatctgcatagaattcaatttcacacgcggttccaaccaggatattatggaaatgactctttcaattattggtccggtaattatgtttcaactagaccaagcataggatcaaatgatataatcacatctccattctatggaaataaatccagtgaacctgtacaaaatttagaatttaatggagaaaaagtctatagagccgtagcaaatacaaatcttgcggtctggccgtccgctgtatattcaggtgttacaaaagtggaatttagccaatataatgatcaaacagatgaagcaagtacacaaacgtacgactcaaaaagaaatgttggcgcggtcagctgggattctatcgatcaattgcctccagaaacaacagatgaacctctagaaaagggatatagccatcaactcaattatgtaatgtgctttttaatgcagggtagtagaggaacaatcccagtgttaacttggacacataaaagtgtagacttttttaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtgctggagataaagtttatatagacaaaattgaatttattccagtgaat

SEQ ID NO: 2 - protein, 644 aa, Bacillus thuringiensis
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 3 - DNA, 1803 bp, Artificial Sequence
  Chemically synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgtctcgagccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagct

SEQ ID NO: 4 - protein, 597 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 5 - DNA, 7208 bp, Artificial Sequence
  Chemically synthesized
gatccaccatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgtctcgagccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagctcaagatctgttgtacaaaaaccagcaactcactgcactgcacttcacttcacttcactgtatgaataaaagtctggtgtctggttcctgatcgatgactgactactccactttgtgcagaacttagtatgtatttgtatttgtaaaatacttctatcaataaaatttctaattcctaaaaccaaaatccagtgggtaccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcacatgacaacaattgtaagaggatggagaccacaacgatccaacaatacttctgcgacgggctgtgaagtatagagaagttaaacgcccaaaagccattgtgtttggaatttttagttattctatttttcatgatgtatcttcctctaacatgccttaatttgcaaatttggtataactactgattgaaaatatatgtatgtaaaaaaatactaagcatatttgtgaagctaaacatgatgttatttaagaaaatatgttgttaacagaataagattaatatcgaaatggaaacatctgtaaattagaatcatcttacaagctaagagatgttcacgctttgagaaacttcttcagatcatgaccgtagaagtagctctccaagactcaacgaaggctgctgcaattccacaaatgcatgacatgcatccttgtaaccgtcgtcgccgctataaacacggataactcaattccctgctccatcaatttagaaatgagcaagcaagcacccgatcgctcaccccatatgcaccaatctgactcccaagtctctgtttcgcattagtaccgccagcactccacctatagctaccaattgagacctttccagcctaagcagatcgattgatcgttagagtcaaagagttggtggtacgggtactttaactaccatggaatgatggggcgtgatgtagagcggaaagcgcctccctacgcggaacaacaccctcgccatgccgctcgactacagcctcctcctcgtcggccgcccacaacgagggagcccgtggtcgcagccaccgaccagcatgtctctgtgtcctcgtccgacctcgacatgtcatggcaaacagtcggacgccagcaccagactgacgacatgagtctctgaagagcccgccacctagaaagatccgagccctgctgctggtagtggtaaccattttcgtcgcgctgacgcggagagcgagaggccagaaatttatagcgactgacgctgtggcaggcacgctatcggaggttacgacgtggcgggtcactcgacgcggagttcacaggtcctatccttgcatcgctcgggccggagtttacgggacttatccttacgacgtgctctaaggttgcgataacgggcggaggaaggcgtgtggcgtgcggagacggtttatacacgtagtgtgcgggagtgtgtttcgtagacgcgggaaagcacgacgacttacgaaggttagtggaggaggaggacacactaaaatcaggacgcaagaaactcttctattatagtagtagagaagagattataggagtgtgggttgattctaaagaaaatcgacgcaggacaaccgtcaaaacgggtgctttaatatagtagatatatatatatagagagagagagaaagtacaaaggatgcatttgtgtctgcatatgatcggagtattactaacggccgtcgtaagaaggtccatcatgcgtggagcgagcccatttggttggttgtcaggccgcagttaaggcctccatatatgattgtcgtcgggcccataacagcatctcctccaccagtttattgtaagaataaattaagtagagatatttgtcgtcgggcagaagaaacttggacaagaagaagaagcaagctaggccaatttcttgccggcaagaggaagatagtggcctctagtttatatatcggcgtgatgatgatgctcctagctagaaatgagagaagaaaaacggacgcgtgtttggtgtgtgtcaatggcgtccatccttccatcagatcagaacgatgaaaaagtcaagcacggcatgcatagtatatgtatagcttgttttagtgtggctttgctgagacgaatgaaagcaacggcgggcatatttttcagtggctgtagctttcaggctgaaagagacgtggcatgcaataattcagggaattcgtcagccaattgaggtagctagtcaacttgtacattggtgcgagcaattttccgcactcaggagggctagtttgagagtccaaaaactataggagattaaagaggctaaaatcctctccttatttaattttaaataagtagtgtatttgtattttaactcctccaacccttccgattttatggctctcaaactagcattcagtctaatgcatgcatgcttggctagaggtcgtatggggttgttaatagcatagctagctacaagttaaccgggtcttttatatttaataaggacaggcaaagtattacttacaaataaagaataaagctaggacgaactcgtggattattactaaatcgaaatggacgtaatattccaggcaagaataattgttcgatcaggagacaagtggggcattggaccggttcttgcaagcaagagcctatggcgtggtgacacggcgcgttgcccatacatcatgcctccatcgatgatccatcctcacttgctataaaaagaggtgtccatggtgctcaagctcagccaagcaaataagacgacttgtttcattgattcttcaagagatcgagcttcttttgcaccacaaggtcgag

SEQ ID NO: 6 - DNA, 1801 bp, Artificial Sequence
  Chemcially synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttcccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagctc

SEQ ID NO: 7 - protein, 596 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 8 - DNA, 1807 bp, Artificial Sequence
  Chemcially synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagctc

SEQ ID NO: 9 - protein, 598 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 10 - DNA, 1818 bp, Artificial Sequence
  Chemcially synthesized
atgaactacaaggagttcctccgcatgaccgccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaac

SEQ ID NO: 11 - protein, 606 aa, Artificial Sequence
  Synthetic Construct
MNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 12 - DNA, 1794 bp, Artificial Sequence
  Chemically synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgtctcgagccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccgctgcaccgttctacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactag

SEQ ID NO: 13 - protein, 597 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPAAPFYFDKTINKGDTLTYNSFNLASFSTPFELSGNNLQIGVTGLSAGDKVYIDKIEFIPVN

SEQ ID NO: 14 - DNA, 1816 bp, Artificial Sequence
  Chemically synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgtctcgagccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatacgctgcaccgttctacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagctc

SEQ ID NO: 15 - protein, 601 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYAAPFYFDKTINKGDTLTYNSFNLASFSTPFELSGNNLQIGVTGLSAGDKVYIDKIEFIPVN

SEQ ID NO: 16 - DNA, 1813 bp, Artificial Sequence
  Chemically synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttcccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatacgctgcaccgttctacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagctc

SEQ ID NO: 17 - protein, 600 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYAAPFYFDKTINKGDTLTYNSFNLASFSTPFELSGNNLQIGVTGLSAGDKVYIDKIEFIPVN

SEQ ID NO: 18 - DNA, 1819 bp, Artificial Sequence
  Chemically synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatacgctgcaccgttctacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgagctc

SEQ ID NO: 19 - protein, 602 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYAAPFYFDKTINKGDTLTYNSFNLASFSTPFELSGNNLQIGVTGLSAGDKVYIDKIEFIPVN

SEQ ID NO: 20 - DNA, 1797 bp, Artificial Sequence
  Chemically synthesized
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccgctgcaccgttctacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactag

SEQ ID NO: 21 - protein, 597 aa, Artificial Sequence
  Synthetic Construct
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPAAPFYFDKTINKGDTLTYNSFNLASFSTPFELSGNNLQIGVTGLSAGDKVYIDKIEFIPV

SEQ ID NO: 22 - DNA, 21 bp, Artificial Sequence
  Chemically synthesized
ggatccaccatgacggccgac

SEQ ID NO: 23 - DNA, 29 bp, Artificial Sequence
  Chemically synthesized
gaacggtgcagcggggttcttctgccagc

SEQ ID NO: 24 - DNA, 29 bp, Artificial Sequence
  Chemically synthesized
gctgcaccgttcccccacagccagggccg

SEQ ID NO: 25 - DNA, 21 bp, Artificial Sequence
  Chemically synthesized
tctagacccacgttgtaccac

SEQ ID NO: 26 - DNA, 29 bp, Artificial Sequence
  Chemically synthesized
gctgcaccgttccgcaacccccacagcca

SEQ ID NO: 27 - DNA, 19 bp, Artificial Sequence
  Chemically synthesized
gagcgtcgacttcttcaac

SEQ ID NO: 28 - DNA, 30 bp, Artificial Sequence
  Chemically synthesized
gaacggtgcagcgtattggttgaagggggc

SEQ ID NO: 29 - DNA, 30 bp, Artificial Sequence
  Chemically synthesized
gctgcaccgttctacttcgacaagaccatc

SEQ ID NO: 30 - DNA, 21 bp, Artificial Sequence
  Chemically synthesized
gagctcagatctagttcacgg

SEQ ID NO: 31 - DNA, 32 bp, Artificial Sequence
  Chemcially synthesized
cggggcccccgctgcaccgttctacttcgaca

SEQ ID NO: 32 - DNA, 32 bp, Artificial Sequence
  Chemically synthesized
tgtcgaagtagaacggtgcagcgggggccccg

SEQ ID NO: 33 - DNA, 48 bp, Artificial Sequence
  Chemically synthesized
ggatccaccatgaactacaaggagttcctccgcatgaccgccgacaac

SEQ ID NO: 34 - DNA, 20 bp, Artificial Sequence
  Chemically synthesized
cctccacctgctccatgaag

SEQ ID NO: 35 - protein, 4 aa, Artificial Sequence
  Protease recognition sequence
AAPF

SEQ ID NO: 36 - protein, 4 aa, Artificial Sequence
  protease recognition sequence
AAPM

SEQ ID NO: 37 - protein, 4 aa, Artificial Sequence
  Protease recognition sequence
AVPF

SEQ ID NO: 38 - protein, 4 aa, Artificial Sequence
  Protease recognition sequence
PFLF

(19) United States
(12) Patent Application Publication
(10) Pub. No.: US 2008/0182796 A1
(43) Pub. Date: Jul. 31, 2008

(54) Modified Cry3A toxins and nucleic acid sequences coding therefor

(75) Inventors: Jeng S. Chen, Research Triangle Park, NC (US); Cheryl Stacy, Research Triangle Park, NC (US); Frederick Walters, Research Triangle Park, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 11/799,009

(22) Filed: Apr. 30, 2007

Related U.S. Application Data
(63) Continuation-in-part of application No. 10/487,846, filed on Feb. 25, 2004, now Pat. No. 7,230,167.
(60) Provisional application No. 60/316,421, filed on Aug. 31, 2001.

(51) Int. Cl. A61K 38/08; C12N 15/31; C12N 15/63; A01H 5/00; A01P 7/00; A61K 38/10; C07K 7/06; C07K 7/08

(52) U.S. Cl. 514/14; 536/23.7; 435/320.1; 435/252.3; 800/298; 800/320.1; 800/302; 530/328; 530/329; 530/327; 514/17; 514/16; 435/69.1; 800/279

(57) ABSTRACT

Compositions and methods for controlling plant pests are disclosed. In particular, novel nucleic acid sequences encoding modified Cry3A toxins having increased toxicity to corn rootworm are provided. By inserting a protease recognition site that is recognized by a gut protease of a target insect in at least one position of a Cry3A toxin a modified Cry3A toxin having significantly greater toxicity, particularly to western and northern corn rootworm is designed. Further, a method of making the modified Cry3A toxins and methods of using the modified cry3A nucleic acid sequences, for example in microorganisms to control insects or in transgenic plants to confer protection from insect damage, and a method of using the modified Cry3A toxins, and compositions and formulations comprising the modified Cry3A toxins, for example applying the modified Cry3A toxins or compositions or formulations to insect-infested areas, or to prophylactically treat insect-susceptible areas or plants to confer protection against the insect pests are disclosed.

The present invention is a continuation-in-part of U.S. application Ser. No. 10/487,846, filed Feb. 25, 2004, currently pending and incorporated herein by reference, which is a 371 of PCT/EP02/09789, filed Feb. 9, 2002, which claims the benefit of U.S. Provisional Application No. 60/316,421, filed Aug. 31, 2001 that is incorporated herein by reference.

The present invention relates to the fields of protein engineering, plant molecular biology and pest control. More particularly, the present invention relates to novel modified Cry3A toxins and nucleic acid sequences whose expression results in the modified Cry3A toxins, and methods of making and methods of using the modified Cry3A toxins and corresponding nucleic acid sequences to control insects.

Species of corn rootworm are considered to be the most destructive corn pests. In the United States the three important species are Diabrotica virgifera virgifera, the western corn rootworm; D. longicornis barberi, the northern corn rootworm and D. undecimpunctata howardi, the southern corn rootworm. Only western and northern corn rootworms are considered primary pests of corn in the US Corn Belt. Corn rootworm larvae cause the most substantial plant damage by feeding almost exclusively on corn roots. This injury has been shown to increase plant lodging, to reduce grain yield and vegetative yield as well as alter the nutrient content of the grain. Larval feeding also causes indirect effects on maize by opening avenues through the roots for bacterial and fungal infections which lead to root and stalk rot diseases. Adult corn rootworms are active in cornfields in late summer where they feed on ears, silks and pollen, interfering with normal pollination.

Corn rootworms are mainly controlled by intensive applications of chemical pesticides, which are active through inhibition of insect growth, prevention of insect feeding or reproduction, or cause death. Good corn rootworm control can thus be reached, but these chemicals can sometimes also affect other, beneficial organisms. Another problem resulting from the wide use of chemical pesticides is the appearance of resistant insect varieties. Yet another problem is due to the fact that corn rootworm larvae feed underground thus making it difficult to apply rescue treatments of insecticides. Therefore, most insecticide applications are made prophylactically at the time of planting. This practice results in a large environmental burden. This has been partially alleviated by various farm management practices, but there is an increasing need for alternative pest control mechanisms.

Biological pest control agents, such as Bacillus thuringiensis (Bt) strains expressing pesticidal toxins like δ-endotoxins, have also been applied to crop plants with satisfactory results against primarily lepidopteran insect pests. The δ-endotoxins are proteins held within a crystalline matrix that are known to possess insecticidal activity when ingested by certain insects. The various δ-endotoxins have been classified based upon their spectrum of activity and sequence homology. Prior to 1990, the major classes were defined by their spectrum of activity with the Cry1 proteins active against Lepidoptera (moths and butterflies), Cry2 proteins active against both Lepidoptera and Diptera (flies and mosquitoes), Cry3 proteins active against Coleoptera (beetles) and Cry4 proteins active against Diptera (Hofte and Whitely, 1989, Microbiol. Rev. 53:242-255). Recently a new nomenclature was developed which systematically classifies the Cry proteins based on amino acid sequence homology rather than insect target specificities (Crickmore et al. 1998, Microbiol. Molec. Biol. Rev. 62:807-813).

The spectrum of insecticidal activity of an individual δ-endotoxin from Bt is quite narrow, with a given δ-endotoxin being active against only a few species within an Order. For instance, the Cry3A protein is known to be very toxic to the Colorado potato beetle, Leptinotarsa decemlineata, but has very little or no toxicity to related beetles in the genus Diabrotica (Johnson et al., 1993, J. Econ. Entomol. 86:330-333). According to Slaney et al. (1992, Insect Biochem. Molec. Biol. 22:9-18) the Cry3A protein is at least 2000 times less toxic to southern corn rootworm larvae than to the Colorado potato beetle. It is also known that Cry3A has little or no toxicity to the western corn rootworm. Specificity of the δ-endotoxins is the result of the efficiency of the various steps involved in producing an active toxin protein and its subsequent interaction with the epithelial cells in the insect mid-gut. To be insecticidal, most known δ-endotoxins must first be ingested by the insect and proteolytically activated to form an active toxin. Activation of the insecticidal crystal proteins is a multi-step process. After ingestion, the crystals must first be solubilized in the insect gut. Once solubilized, the δ-endotoxins are activated by specific proteolytic cleavages. The proteases in the insect gut can play a role in specificity by determining where the δ-endotoxin is processed. Once the δ-endotoxin has been solubilized and processed it binds to specific receptors on the surface of the insects' mid-gut epithelium and subsequently integrates into the lipid bilayer of the brush border membrane. Ion channels then form disrupting the normal function of the midgut eventually leading to the death of the insect.

In Lepidoptera, gut proteases process 6-endotoxins from 130-140 kDa protoxins to toxic proteins of approximately 60-70 kDa. Processing of the protoxin to toxin has been reported to proceed by removal of both N— and C-terminal amino acids with the exact location of processing being dependent on the specific insect gut fluids involved (Ogiwara et al., 1992, J. Invert. Pathol. 60:121-126). The proteolytic activation of a δ-endotoxin can play a significant role in determining its specificity. For example, a δ-endotoxin from Bt var. aizawa, called IC1, has been classified as a Cry1Ab protein based on its sequence homology with other known Cry1Ab proteins. Cry1Ab proteins are typically active against lepidopteran insects. However, the IC1 protein has activity against both lepidopteran and dipteran insects depending upon how the protein is processed (Haider et al. 1986, Euro. J. Biochem. 156: 531-540). In a dipteran gut, a 53 kDa active IC1 toxin is obtained, whereas in a lepidopteran gut, a 55 kDa active IC1 toxin is obtained. IC1 differs from the holotype HD-1 Cry1Ab protein by only four amino acids, so gross changes in the receptor binding region do not seem to account for the differences in activity. The different proteolytic cleavages in the two different insect guts possibly allow the activated molecules to fold differently thus exposing different regions capable of binding different receptors. The specificity therefore, appears to reside with the gut proteases of the different insects. Coleopteran insects have guts that are more neutral to acidic and coleopteran-specific δ-endotoxins are similar to the size of the activated lepidopteran-specific toxins. Therefore, the processing of coleopteran-specific δ-endotoxins was formerly considered unnecessary for toxicity. However, recent data suggests that coleopteran-active δ-endotoxins are solubilized and proteolyzed to smaller toxic polypeptides. The 73 kDa Cry3A δ-endotoxin protein produced by B. thuringiensis var. tenebrionis is readily processed in the bacterium at the N-terminus, losing 49-57 residues during or after crystal formation to produce the commonly isolated 67 kDa form (Carroll et al., 1989, Biochem. J. 261:99-105). McPherson et al., 1988 (Biotechnology 6:61-66) also demonstrated that the native cry3A gene contains two functional translational initiation codons in the same reading frame, one coding for the 73 kDa protein and the other coding for the 67 kDa protein starting at Met-1 and Met-48 respectively, of the deduced amino acid sequence (See SEQ ID NO: 2). Both proteins then can be considered naturally occurring full-length Cry3A proteins. Treatment of soluble 67 kDa Cry3A protein with either trypsin or insect gut extract results in a cleavage product of 55 kDa with Asn-159 of the deduced amino acid sequence at the N-terminus. This polypeptide was found to be as toxic to a susceptible coleopteran insect as the native 67 kDa Cry3A toxin. (Carroll et al. Ibid). Thus, a natural trypsin recognition site exists between Arg-158 and Asn-159 of the deduced amino acid sequence of the native Cry3A toxin (SEQ ID NO: 2). Cry3A can also be cleaved by chymotrypsin, resulting in three polypeptides of 49, 11, and 6 kDa. N-terminal analysis of the 49 and 6 kDa components showed the first amino acid residue to be Ser-162 and Tyr-588, respectively (Carroll et al., 1997 J. Invert. Biol. 70:41-49). Thus, natural chymotrypsin recognition sites exist in Cry3A between His-161 and Ser-162 and between Tyr-587 and Tyr-588 of the deduced amino acid sequence (SEQ ID NO: 2). The 49 kDa chymotrypsin product appears to be more soluble at neutral pH than the native 67 kDa protein or the 55 kDa trypsin product and retains full insecticidal activity against the Cry3A-susceptible insects, Colorado potato beetle and mustard beetle, (Phaedon cochleariae).

Insect gut proteases typically function in aiding the insect in obtaining needed amino acids from dietary protein. The best understood insect digestive proteases are serine proteases that appear to be the most common (Englemann and Geraerts, 1980, J. Insect Physiol. 261:703-710), particularly in lepidopteran species. The majority of coleopteran larvae and adults, for example Colorado potato beetle, have slightly acidic midguts, and cysteine proteases provide the major proteolytic activity (Wolfson and Mudock, 1990, J. Chem. Ecol. 16:1089-1102). More precisely, Thie and Houseman (1990, Insect Biochem. 20:313-318) identified and characterized the cysteine proteases, cathepsin B and H, and the aspartyl protease, cathepsin D in Colorado potato beetle. Gillikin et al. (1992, Arch. Insect Biochem. Physiol. 19:285-298) characterized the proteolytic activity in the guts of western corn rootworm larvae and found 15, primarily cysteine, proteases. Until disclosed in this invention, no reports have indicated that the serine protease, cathepsin G, exists in western corn rootworm. The diversity and different activity levels of the insect gut proteases may influence an insect's sensitivity to a particular Bt toxin.

Many new and novel Bt strains and δ-endotoxins with improved or novel biological activities have been described over the past five years including strains active against nematodes (EP 0517367A1). However, relatively few of these strains and toxins have activity against coleopteran insects. Further, none of the now known coleopteran-active δ-endotoxins, for example Cry3A, Cry3B, Cry3C, Cry7A, Cry8A, Cry8B, and Cry8C, have sufficient oral toxicity against corn rootworm to provide adequate field control if delivered, for example, through microbes or transgenic plants. Therefore, other approaches for producing novel toxins active against corn rootworm need to be explored. As more knowledge has been gained as to how the δ-endotoxins function, attempts to engineer δ-endotoxins to have new activities have increased. Engineering δ-endotoxins was made more possible by the solving of the three dimensional structure of Cry3A in 1991 (Li et al., 1991, Nature 353:815-821). The protein has three structural domains: the N-terminal domain I, from residues 1-290, consists of 7 alpha helices, domain II, from residues 291-500, contains three beta-sheets and the C-terminal domain III, from residues 501-644, is a beta-sandwich. Based on this structure, a hypothesis has been formulated regarding the structure/function relationship of the δ-endotoxins. It is generally thought that domain I is primarily responsible for pore formation in the insect gut membrane (Gazit and Shai, 1993, Appl. Environ. Microbiol. 57:2816-2820), domain II is primarily responsible for interaction with the gut receptor (Ge et al., 1991, J. Biol. Chem. 32:3429-3436) and that domain III is most likely involved with protein stability (Li et al. 1991, supra) as well as having a regulatory impact on ion channel activity (Chen et al., 1993, PNAS 90:9041-9045).

Lepidopteran-active δ-endotoxins have been engineered in attempts to improve specific activity or to broaden the spectrum of insecticidal activity. For example, the silk moth (Bombyx mori) specificity domain from Cry1Aa was moved to Cry1Ac, thus imparting a new insecticidal activity to the resulting chimeric protein (Ge et al. 1989, PNAS 86: 4037-4041). Also, Bosch et al. 1998 (U.S. Pat. No. 5,736,131), created a new lepidopteran-active toxin by substituting domain III of Cry1E with domain III of Cry1C thus producing a Cry1E-Cry1C hybrid toxin with a broader spectrum of lepidopteran activity. Several attempts at engineering the coleopteran-active δ-endotoxins have been reported. Van Rie et al., 1997, (U.S. Pat. No. 5,659,123) engineered Cry3A by randomly replacing amino acids, thought to be important in solvent accessibility, in domain II with the amino acid alanine. Several of these random replacements confined to receptor binding domain II were reportedly involved in increased western corn rootworm toxicity. However, others have shown that some alanine replacements in domain II of Cry3A result in disruption of receptor binding or structural instability (Wu and Dean, 1996, J. Mol. Biol. 255: 628-640). English et al., 1999, (Intl. Pat. Appl. Publ. No. WO 99/31248) reported amino acid substitutions in Cry3Bb that caused increases in toxicity to southern and western corn rootworm. However, of the 35 reported Cry3Bb mutants, only three, with mutations primarily in domain II and the domain II-domain I interface, were active against western corn rootworm. Further, the differences in toxicity of wild-type Cry3Bb against western corn rootworm in the same assays were greater than any of the differences between the mutated Cry3Bb toxins and the wild-type Cry3Bb. Therefore, improvements in toxicity of the Cry3Bb mutants appear to be confined primarily to southern corn rootworm. There remains a need to design new and effective pest control agents that provide an economic benefit to farmers and that are environmentally acceptable. Particularly needed are modified Cry3A toxins that control western corn rootworm, the major pest of corn in the United States, that are or could become resistant to existing insect control agents. Furthermore, agents whose application minimizes the burden on the environment, as through transgenic plants, are desirable.

In view of these needs, it is an object of the present invention to provide novel nucleic acid sequences encoding modified Cry3A toxins having increased toxicity to corn rootworm. By inserting a protease recognition site that is recognized by a target-insect gut protease in at least one position of a Cry3A toxin, in accordance with the present invention, a modified Cry3A toxin having significantly greater toxicity, particularly to western and northern corn rootworm is designed. The invention is further drawn to the novel modified Cry3A toxins resulting from the expression of the nucleic acid sequences, and to compositions and formulations containing the modified Cry3A toxins, which are capable of inhibiting the ability of insect pests to survive, grow and reproduce, or of limiting insect-related damage or loss to crop plants. The invention is further drawn to a method of making the modified Cry3A toxins and to methods of using the modified cry3A nucleic acid sequences, for example in microorganisms to control insects or in transgenic plants to confer protection from insect damage, and to a method of using the modified Cry3A toxins, and compositions and formulations comprising the modified Cry3A toxins, for example applying the modified Cry3A toxins or compositions or formulations to insect-infested areas, or to prophylactically treat insect-susceptible areas or plants to confer protection against the insect pests.

The novel modified Cry3A toxins described herein are highly active against insects. For example, the modified Cry3A toxins of the present invention can be used to control economically important insect pests such as western corn rootworm (Diabrotica virgifera virgifera) and northern corn rootworm (D. longicornis barberi). The modified Cry3A toxins can be used singly or in combination with other insect control strategies to confer maximal pest control efficiency with minimal environmental impact.

According to one aspect, the present invention provides an isolated nucleic acid molecule comprising a nucleotide sequence that encodes a modified Cry3A toxin, wherein the modified Cry3A toxin comprises at least one additional protease recognition site that does not naturally occur in a Cry3A toxin. The additional protease recognition site, which is recognized by a gut protease of a target insect, is inserted at approximately the same position as a naturally occurring protease recognition site in the Cry3A toxin. The modified Cry3A toxin causes higher mortality to a target insect than the mortality caused by a Cry3A toxin to the same target insect. Preferably, the modified Cry3A toxin causes at least about 50% mortality to a target insect to which a Cry3A toxin causes only up to about 30% mortality.

In one embodiment of this aspect, the gut protease of a target insect is selected from the group consisting of serine proteases, cysteine proteases and aspartic proteases. Preferable serine proteases according to this embodiment include cathepsin G, trypsin, chymotrypsin, carboxypeptidase, endopeptidase and elastase, most preferably cathepsin G.

In another embodiment of this aspect, the additional protease recognition site is inserted in either domain I or domain III or in both domain I and domain III of the Cry3A toxin. Preferably, the additional protease recognition site is inserted in either domain I or domain III or in both domain I and domain III at a position that replaces, is adjacent to, or is within a naturally occurring protease recognition site.

In a yet another embodiment, the additional protease recognition site is inserted in domain I between amino acids corresponding to amino acid numbers 154 and 162 of SEQ ID NO: 2. Preferably, the additional protease recognition site is inserted between amino acid numbers 154 and 162 of SEQ ID NO: 2 or between amino acid numbers 107 and 115 of SEQ ID NO: 4.

In still another embodiment, the additional protease recognition site is inserted between amino acids corresponding to amino acid numbers 154 and 160 of SEQ ID NO: 2. Preferably, the additional protease recognition site is inserted between amino acid numbers 154 and 160 of SEQ ID NO: 2 or between amino acid numbers 107 and 113 of SEQ ID NO: 4.

In a further embodiment, the additional protease recognition site is inserted in domain I between amino acids corresponding to amino acid numbers 154 and 158 of SEQ ID NO: 2. Preferably, the additional protease recognition site is inserted in domain I between amino acid numbers 154 and 158 of SEQ ID NO: 2 or between amino acid numbers 107 and 111 of SEQ ID NO: 4.

In another embodiment, the additional protease recognition site is inserted in domain III between amino acids corresponding to amino acid numbers 583 and 589 of SEQ ID NO: 2. Preferably, the additional protease site is inserted in domain III between amino acid numbers 583 and 589 of SEQ ID NO: 2 or between amino acid numbers 536 and 542 of SEQ ID NO: 4.

In still another embodiment, the additional protease recognition site is inserted in domain III between amino acids corresponding to amino acid numbers 583 and 588 of SEQ ID NO: 2. Preferably, the additional protease site is inserted in domain III between amino acid numbers 583 and 588 of SEQ ID NO: 2 or between amino acid numbers 536 and 541 of SEQ ID NO: 4.

In yet another embodiment, the additional protease recognition site is inserted in domain III between amino acids corresponding to amino acid numbers 587 and 588 of SEQ ID NO: 2. Preferably, the additional protease site is inserted in domain III between amino acid numbers 587 and 588 of SEQ ID NO: 2 or between amino acid numbers 540 and 541 of SEQ ID NO: 4.

In one embodiment, the additional protease recognition site is inserted in domain I and domain III of the unmodified Cry3A toxin. Preferably, the additional protease recognition site is inserted in domain I at a position that replaces or is adjacent to a naturally occurring protease recognition site and in domain III at a position that is within, replaces, or is adjacent to a naturally occurring protease recognition site.

In another embodiment, the additional protease recognition site is inserted in domain I between amino acids corresponding to amino acid numbers 154 and 160 and in domain III between amino acids corresponding to amino acid numbers 587 and 588 of SEQ ID NO: 2.

Preferably, the additional protease recognition site is inserted in domain I between amino acid numbers 154 and 160 and in domain III between amino acid numbers 587 and 588 of SEQ ID NO: 2 or in domain I between amino acid numbers 107 and 113 and in domain III between amino acid numbers 540 and 541 of SEQ ID NO: 4.

In yet another embodiment, the additional protease recognition site is located in domain I between amino acids corresponding to amino acid numbers 154 and 158 and in domain III between amino acids corresponding to amino acid numbers 587 and 588 of SEQ ID NO: 2.

Preferably, the additional protease recognition site is inserted in domain I between amino acid numbers 154 and 158 and in domain III between amino acid numbers 587 and 588 of SEQ ID NO: 2 or in domain I between amino acid numbers 107 and 111 and in domain III between amino acid numbers 540 and 541 of SEQ ID NO: 4.

In another embodiment, the additional protease recognition site is located in domain I between amino acids corresponding to amino acid numbers 154 and 158 and in domain III between amino acids corresponding to amino acid numbers 583 and 588 of SEQ ID NO: 2.

Preferably, the additional protease recognition site is inserted in domain I between amino acid numbers 154 and 158 and in domain III between amino acid numbers 583 and 588 of SEQ ID NO: 2 or in domain I between amino acid numbers 107 and 111 and in domain III between amino acid numbers 536 and 541 of SEQ ID NO: 4.

In a preferred embodiment, the isolated nucleic acid molecule of the present invention comprises nucleotides 1-1791 of SEQ ID NO: 6, nucleotides 1-1806 of SEQ ID NO: 8, nucleotides 1-1818 of SEQ ID NO: 10, nucleotides 1-1794 of SEQ ID NO: 12, nucleotides 1-1812 of SEQ ID NO: 14, nucleotides 1-1812 of SEQ ID NO: 16, nucleotides 1-1818 of SEQ ID NO: 18, or nucleotides 1-1791 of SEQ ID NO: 20.

In another preferred embodiment, the isolated nucleic acid molecule of the invention encodes a modified Cry3A toxin comprising the amino acid sequence set forth in SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 21.

According to one embodiment of the invention, the isolated nucleic acid molecule encodes a modified Cry3A toxin that is active against a coleopteran insect. Preferably, the modified Cry3A toxin has activity against western corn rootworm.

The present invention provides a chimeric gene comprising a heterologous promoter sequence operatively linked to the nucleic acid molecule of the invention. The present invention also provides a recombinant vector comprising such a chimeric gene. Further, the present invention provides a transgenic non-human host cell comprising such a chimeric gene. A transgenic host cell according to this aspect of the invention may be a bacterial cell or a plant cell, preferably, a plant cell. The present invention further provides a transgenic plant comprising such a plant cell. A transgenic plant according to this aspect of the invention may be sorghum, wheat, sunflower, tomato, potato, cole crops, cotton, rice, soybean, sugar beet, sugarcane, tobacco, barley, oilseed rape, or maize, preferably, maize. The present invention also provides seed from the group of transgenic plants consisting of sorghum, wheat, sunflower, tomato, potato, cole crops, cotton, rice, soybean, sugar beet, sugarcane, tobacco, barley, oilseed rape, and maize. In a particularly preferred embodiment, the seed is from a transgenic maize plant.

In another aspect, the present invention provides toxins produced by the expression of the nucleic acid molecules of the present invention. In a preferred embodiment, the toxin is produced by the expression of the nucleic acid molecule comprising nucleotides 1-1791 of SEQ ID NO: 6, nucleotides 1-1806 of SEQ ID NO: 8, nucleotides 1-1818 of SEQ ID NO: 10, nucleotides 1-1794 of SEQ ID NO: 12, nucleotides 1-1812 of SEQ ID NO: 14, nucleotides 1-1812 of SEQ ID NO: 16, nucleotides 1-1818 of SEQ ID NO: 18, or nucleotides 1-1791 of SEQ ID NO: 20.

In another embodiment, the toxins of the invention are active against coleopteran insects, preferably against western corn rootworm.

In one embodiment, a toxin of the present invention comprises the amino acid sequence set forth in SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 21.

The present invention also provides a composition comprising an effective insect-controlling amount of a toxin according to the invention.

In another aspect, the present invention provides a method of producing a toxin that is active against insects, comprising: (a) obtaining a host cell comprising a chimeric gene, which itself comprises a heterologous promoter sequence operatively linked to the nucleic acid molecule of the invention; and (b) expressing the nucleic acid molecule in the transgenic host cell, which results in at least one toxin that is active against insects.

In a further aspect, the present invention provides a method of producing an insect-resistant transgenic plant, comprising introducing a nucleic acid molecule of the invention into the transgenic plant, wherein the nucleic acid molecule is expressible in the transgenic plant in an effective amount to control insects. In a preferred embodiment, the insects are coleopteran insects, preferably western corn rootworm.

In yet a further aspect, the present invention provides a method of controlling insects, comprising delivering to the insects an effective amount of a toxin of the invention. According to one embodiment, the insects are coleopteran insects, preferably, western corn rootworm.

Preferably, the toxin is delivered to the insects orally. In one preferred embodiment, the toxin is delivered orally through a transgenic plant comprising a nucleic acid sequence that expresses a toxin of the present invention.

Also provided by the present invention is a method of making a modified Cry3A toxin, comprising: (a) obtaining a cry3A toxin gene which encodes a Cry3A toxin; (b) identifying a gut protease of a target insect; (c) obtaining a nucleotide sequence which encodes a recognition sequence for the gut protease; (d) inserting the nucleotide sequence of (c) into either domain I or domain III or both domain I and domain III at a position that replaces, is within, or adjacent to a nucleotide sequence that codes for a naturally occurring protease recognition site in a cry3A toxin gene, thus creating a modified cry3A toxin gene; (e) inserting the modified cry3A toxin gene in an expression cassette; (f) expressing the modified cry3A toxin gene in a non-human host cell, resulting in the host cell producing a modified Cry3A toxin; and, (g) bioassaying the modified Cry3A toxin against a target insect, whereby the modified Cry3A toxin causes higher mortality to the target insect than the mortality caused by a Cry3A toxin. In a preferred embodiment, the modified Cry3A toxin causes at least about 50% mortality to the target insect when the Cry3A toxin causes up to about 30% mortality.

The present invention further provides a method of controlling insects wherein the transgenic plant further comprises a second nucleic acid sequence or groups of nucleic acid sequences that encode a second pesticidal principle. Particularly preferred second nucleic acid sequences are those that encode a δ-endotoxin, those that encode a Vegetative Insecticidal Protein toxin, disclosed in U.S. Pat. Nos. 5,849,870 and 5,877,012, incorporated herein by reference, or those that encode a pathway for the production of a non-proteinaceous pesticidal principle.

Yet another aspect of the present invention is the provision of a method for mutagenizing a nucleic acid molecule according to the present invention, wherein the nucleic acid molecule has been cleaved into populations of double-stranded random fragments of a desired size, comprising: (a) adding to the population of double-stranded random fragments one or more single- or double-stranded oligonucleotides, wherein the oligonucleotides each comprise an area of identity and an area of heterology to a double-stranded template polynucleotide; (b) denaturing the resultant mixture of double-stranded random fragments and oligonucleotides into single-stranded fragments; (c) incubating the resultant population of single-stranded fragments with polymerase under conditions which result in the annealing of the single-stranded fragments at the areas of identity to form pairs of annealed fragments, the areas of identity being sufficient for one member of the pair to prime replication of the other, thereby forming a mutagenized double-stranded polynucleotide; and (d) repeating the second and third steps for at least two further cycles, wherein the resultant mixture in the second step of a further cycle includes the mutagenized double-stranded polynucleotide from the third step of the previous cycle, and wherein the further cycle forms a further mutagenized double-stranded polynucleotide.

Other aspects and advantages of the present invention will become apparent to those skilled in the art from a study of the following description of the invention and non-limiting examples.

SEQ ID NO: 1 is the native cry3A coding region.

SEQ ID NO: 2 is the amino acid sequence of the Cry3A toxin encoded by the native cry3A gene.

SEQ ID NO: 3 is the maize optimized cry3A coding region beginning at nucleotide 144 of the native cry3A coding region.

SEQ ID NO: 4 is the amino acid sequence of the Cry3A toxin encoded by the maize optimized cry3A gene.

SEQ ID NO: 5 is the nucleotide sequence of pCIB6850.

SEQ ID NO: 6 is the maize optimized modified cry3A054 coding sequence.

SEQ ID NO: 7 is the amino acid sequence encoded by the nucleotide sequence of SEQ ID NO: 6.

SEQ ID NO: 8 is the maize optimized modified cry3A055 coding sequence.

SEQ ID NO: 9 is the amino acid sequence encoded by the nucleotide sequence of SEQ ID NO: 8.

SEQ ID NO: 10 is the maize optimized modified cry3A085 coding sequence.

SEQ ID NO: 11 is the amino acid sequence encoded by the nucleotide sequence of SEQ ID NO: 10.

SEQ ID NO: 12 is the maize optimized modified cry3A082 coding sequence.

SEQ ID NO: 13 is the amino acid sequence encoded by the nucleotide sequence of SEQ ID NO: 12.

SEQ ID NO: 14 is the maize optimized modified cry3A058 coding sequence.

SEQ ID NO: 15 is the amino acid sequence encoded by the nucleotide sequence of SEQ ID NO: 14.

SEQ ID NO: 16 is the maize optimized modified cry3A057 coding sequence.

SEQ ID NO: 17 is the amino acid sequence encoded by the nucleotide sequence of SEQ ID NO: 16.

SEQ ID NO: 18 is the maize optimized modified cry3A056 coding sequence.

SEQ ID NO: 19 is the amino acid sequence encoded by the nucleotide sequence of SEQ ID NO: 18.

SEQ ID NO: 20 is the maize optimized modified cry3A083 coding sequence.

SEQ ID NO: 21 is the amino acid sequence encoded by the nucleotide sequence of SEQ ID NO: 20.

SEQ ID NOS: 22-34 are PCR primers useful in the present invention.

SEQ ID NO: 35 is an amino acid sequence comprising a cathepsin G recognition site.

SEQ ID NO: 36 is an amino acid sequence comprising a cathepsin G recognition site.

SEQ ID NO: 37 is an amino acid sequence comprising a cathepsin G recognition site.

SEQ ID NO: 38 is an amino acid sequence comprising a cathepsin G recognition site.

For clarity, certain terms used in the specification are defined and presented as follows: “Activity” of the modified Cry3A toxins of the invention is meant that the modified Cry3A toxins function as orally active insect control agents, have a toxic effect, or are able to disrupt or deter insect feeding, which may or may not cause death of the insect. When a modified Cry3A toxin of the invention is delivered to the insect, the result is typically death of the insect, or the insect does not feed upon the source that makes the modified Cry3A toxin available to the insect.

“Adjacent to”—According to the present invention, an additional protease recognition site is “adjacent to” a naturally occurring protease recognition site when the additional protease recognition site is within four residues, preferably within three residues, more preferably within two residues, and most preferably within one residue of a naturally occurring protease recognition site. For example, an additional protease recognition site inserted between Pro-154 and Arg-158 of the deduced amino acid sequence of a Cry3A toxin (SEQ ID NO: 2) is “adjacent to” the naturally occurring trypsin recognition site located between Arg-158 and Asn-159 of the deduced amino acid sequence of the Cry3A toxin (SEQ ID NO: 2).

The phrase “approximately the same position” as used herein to describe the location where an additional protease recognition site is inserted into a Cry3A toxin in relation to a naturally occurring protease recognition site, means that the location is at most four residues away from a naturally occurring protease recognition site. The location can also be three or two residues away from a naturally occurring protease recognition site. The location can also be one residue away from a naturally occurring protease recognition site. “Approximately the same position” can also mean that the additional protease recognition site is inserted within a naturally occurring protease recognition site.

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulatory DNA sequence will affect the expression level of the coding or structural DNA sequence.

A “chimeric gene” or “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid coding sequence. The regulatory nucleic acid sequence of the chimeric gene is not normally operatively linked to the associated nucleic acid sequence as found in nature.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

To “control” insects means to inhibit, through a toxic effect, the ability of insect pests to survive, grow, feed, and/or reproduce, or to limit insect-related damage or loss in crop plants. To “control” insects may or may not mean killing the insects, although it preferably means killing the insects.

Corresponding to: in the context of the present invention, “corresponding to” means that when the amino acid sequences of variant Cry3A δ-endotoxins are aligned with each other, the amino acids that “correspond to” certain enumerated positions in the present invention are those that align with these positions in the Cry3A toxin (SEQ ID NO: 2), but that are not necessarily in these exact numerical positions relative to the particular Cry3A amino acid sequence of the invention. For example, the maize optimized cry3A gene (SEQ ID NO: 3) of the invention encodes a Cry3A toxin (SEQ ID NO: 4) that begins at Met-48 of the Cry3A toxin (SEQ ID NO: 2) encoded by the native cry3A gene (SEQ ID NO: 1).

Therefore, according to the present invention, amino acid numbers 107-115, including all numbers in between, and 536-541, including all numbers in between, of SEQ ID NO: 4 correspond to amino acid numbers 154-163, and all numbers in between, and 583-588, and all numbers in between, respectively, of SEQ ID NO: 2.

A “Cry3A toxin”, as used herein, refers to an approximately 73 kDa Bacillus thuringiensis var. tenebrionis (Kreig et al., 1983, Z. Angew. Entomol. 96:500-508) (Bt) coleopteran-active protein (Sekar et al., 1987, Proc. Nalt. Acad. Sci. 84:7036-7040), for example SEQ ID NO: 2, as well as any truncated lower molecular weight variants, derivable from a Cry3A toxin, for example SEQ ID NO: 4, and retaining substantially the same toxicity as the Cry3A toxin. The lower molecular weight variants can be obtained by protease cleavage of naturally occurring protease recognition sites of the Cry3A toxin or by a second translational initiation codon in the same frame as the translational initiation codon coding for the 73 kDa Cry3A toxin. The amino acid sequence of a Cry3A toxin and the lower molecular weight variants thereof can be found in a toxin naturally occurring in Bt.

A Cry3A toxin can be encoded by a native Bt gene as in SEQ ID NO: 1 or by a synthetic coding sequence as in SEQ ID NO: 3. A “Cry3A toxin” does not have any additional protease recognition sites over the protease recognition sites that naturally occur in the Cry3A toxin. A Cry3A toxin can be isolated, purified or expressed in a heterologous system.

A “cry3A gene”, as used herein, refers to the nucleotide sequence of SEQ ID NO: 1 or SEQ ID NO: 3. A cry3A gene (Sekar et al., 1987, Proc. Natl. Acad. Sci. 84:7036-7040) can be naturally occurring, as found in Bacillus thuringiensis var. tenebrionis (Kreig et al., 1983, Z. Angew. Entomol. 96:500-508), or synthetic and encodes a Cry3A toxin. The cry3A gene of this invention can be referred to as the native cry3A gene as in SEQ ID NO: 1 or the maize-optimized cry3A gene as in SEQ ID NO: 3.

To “deliver” a toxin means that the toxin comes in contact with an insect, resulting in toxic effect and control of the insect. The toxin can be delivered in many recognized ways, e.g., orally by ingestion by the insect or by contact with the insect via transgenic plant expression, formulated protein composition(s), sprayable protein composition(s), a bait matrix, or any other art-recognized toxin delivery system.

“Effective insect-controlling amount” means that concentration of toxin that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns.

“Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

A “gut protease” is a protease naturally found in the digestive tract of an insect. This protease is usually involved in the digestion of ingested proteins.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

“Homologous recombination” is the reciprocal exchange of nucleic acid fragments between homologous nucleic acid molecules.

“Insecticidal” is defined as a toxic biological activity capable of controlling insects, preferably by killing them.

A nucleic acid sequence is “isocoding with” a reference nucleic acid sequence when the nucleic acid sequence encodes a polypeptide having the same amino acid sequence as the polypeptide encoded by the reference nucleic acid sequence.

An “isolated” nucleic acid molecule or an isolated toxin is a nucleic acid molecule or toxin that, by the hand of man, exists apart from its native environment and is therefore not a product of nature. An isolated nucleic acid molecule or toxin may exist in a purified form or may exist in a non-native environment such as, for example, a recombinant host cell.

A “modified Cry3A toxin” of this invention, refers to a Cry3A-derived toxin having at least one additional protease recognition site that is recognized by a gut protease of a target insect, which does not naturally occur in a Cry3A toxin. A modified Cry3A toxin is not naturally occurring and, by the hand of man, comprises an amino acid sequence that is not identical to a naturally occurring toxin found in Bacillus thuringiensis. The modified Cry3A toxin causes higher mortality to a target insect than the mortality caused by a Cry3A toxin to the same target insect.

A “modified cry3A gene” according to this invention, refers to a cry3A-derived gene comprising the coding sequence of at least one additional protease recognition site that does not naturally occur in an unmodified cry3A gene. The modified cry3A gene can be derived from a native cry3A gene or from a synthetic cry3A gene.

A “naturally occurring protease recognition site” is a location within a Cry3A toxin that is cleaved by a non-insect derived protease or by a protease or gut extract from an insect species susceptible to the Cry3A toxin. For example, a naturally occurring protease recognition site, recognized by trypsin and proteases found in a susceptible insect gut extract, exists between Arg-158 and Asn-159 of the deduced Cry3A toxin amino acid sequence (SEQ ID NO: 2). Naturally occurring protease recognition sites, recognized by chymotrypsin, exist between His-161 and Ser-162 as well as between Tyr-587 and Tyr-588 of the deduced Cry3A toxin amino acid sequence (SEQ ID NO: 2).

A “nucleic acid molecule” or “nucleic acid sequence” is a linear segment of single- or double-stranded DNA or RNA that can be isolated from any source. In the context of the present invention, the nucleic acid molecule is preferably a segment of DNA.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant. A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A “promoter” is an untranslated DNA sequence upstream of the coding region that contains the binding site for RNA polymerase and initiates transcription of the DNA. The promoter region may also include other elements that act as regulators of gene expression. A “protoplast” is an isolated plant cell without a cell wall or with only parts of the cell wall.

“Regulatory elements” refer to sequences involved in controlling the expression of a nucleotide sequence. Regulatory elements comprise a promoter operably linked to the nucleotide sequence of interest and termination signals. They also typically encompass sequences required for proper translation of the nucleotide sequence.

“Replaces” a naturally occurring protease recognition site—According to the present invention, an additional protease recognition site “replaces” a naturally occurring protease recognition site when insertion of the additional protease recognition site eliminates the naturally occurring protease recognition site. For example, an additional protease recognition site inserted between Pro-154 and Pro-160 of the deduced amino acid sequence of a Cry3A toxin (SEQ ID NO: 2) which eliminates the Arg-158 and Asn-159 residues “replaces” the naturally occurring trypsin recognition site located between Arg-158 and Asn-159 of the deduced amino acid sequence of the Cry3A toxin (SEQ ID NO: 2).

“Serine proteases”, describe the same group of enzymes that catalyze the hydrolysis of covalent peptidic bonds using a mechanism based on nucleophilic attack of the targeted peptidic bond by a serine. Serine proteases are sequence specific. That is, each serine protease recognizes a specific sub-sequence within a protein where enzymatic recognition occurs.

A “target insect” is an insect pest species that has little or no susceptibility to a Cry3A toxin and is identified as being a candidate for using the technology of the present invention to control. This control can be achieved through several means but most preferably through the expression of the nucleic acid molecules of the invention in transgenic plants.

A “target insect gut protease” is a protease found in the gut of a target insect whose recognition site can be inserted into a Cry3A toxin to create a modified Cry3A toxin of the invention.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

“Within” a naturally occurring protease recognition site—According to the present invention, an additional protease recognition site is “within” a naturally occurring protease recognition site when the additional protease recognition site lies between the amino acid residue that comes before and the amino acid residue that comes after the naturally occurring protease recognition site. For example, an additional protease recognition site inserted between Tyr-587 and Tyr-588 of the deduced amino acid sequence of a Cry3A toxin (SEQ ID NO: 2) is “within” a naturally occurring chymotrypsin recognition site located between Tyr-587 and Tyr-588 of the deduced amino acid sequence of the Cry3A toxin (SEQ ID NO: 2). The insertion of an additional protease recognition site within a naturally occurring protease recognition site may or may not change the recognition of the naturally occurring protease recognition site by a protease.

Nucleotides are indicated by their bases by the following standard abbreviations: adenine (A), cytosine (C), thymine (T), and guanine (G). Amino acids are likewise indicated by the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (lie; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

This invention relates to modified cry3A nucleic acid sequences whose expression results in modified Cry3A toxins, and to the making and using of the modified Cry3A toxins to control insect pests. The expression of the modified cry3A nucleic acid sequences results in modified Cry3A toxins that can be used to control coleopteran insects such as western corn rootworm and northern corn rootworm. A modified Cry3A toxin of the present invention comprises at least one additional protease recognition site that does not naturally occur in a Cry3A toxin. The additional protease recognition site, which is recognized by a gut protease of a target insect, is inserted at approximately the same position as a naturally occurring protease recognition site in a Cry3A toxin. The modified Cry3A toxin causes higher mortality to a target insect than the mortality caused by a Cry3A toxin to the same target insect. Preferably, the modified Cry3A toxin causes at least about 50% mortality to the target insect to which a Cry3A toxin causes up to about 30% mortality.

In one preferred embodiment, the invention encompasses an isolated nucleic acid molecule that encodes a modified Cry3A toxin, wherein the additional protease recognition site is recognized by the target insect gut protease, cathepsin G. Cathepsin G activity is determined to be present in the gut of the target insect, western corn rootworm, as described in Example 2. Preferably, the substrate amino acid sequence, AAPF (SEQ ID NO: 35), used to determine the presence of the cathepsin G activity is inserted into the Cry3A toxin according to the present invention. Other cathepsin G recognition sites can also be used according to the present invention, for example, AAPM (SEQ ID NO: 36), AVPF (SEQ ID NO: 37), PFLF (SEQ ID NO: 38) or other cathepsin G recognition sites as determined by the method of Tanaka et al., 1985 (Biochemistry 24:2040-2047), incorporated herein by reference. Protease recognition sites of other proteases identified in a target insect gut can be used, for example, protease recognition sites recognized by other serine proteases, cysteine proteases and aspartic proteases. Preferable serine proteases encompassed by this embodiment include trypsin, chymotrypsin, carboxypeptidase, endopeptidase and elastase.

In another preferred embodiment, the invention encompasses an isolated nucleic acid molecule that encodes a modified Cry3A toxin wherein the additional protease recognition site is inserted in either domain I or domain III or in both domain I and domain III of the Cry3A toxin. Preferably, the additional protease recognition site is inserted in domain I, domain III, or domain I and domain III at a position that replaces, is adjacent to, or is within a naturally occurring protease recognition site in the Cry3A toxin. Specifically exemplified herein are nucleic acid molecules that encode modified Cry3A toxins that comprise a cathepsin G recognition site inserted in domain I, domain III, or domain I and domain III at a position that replaces, is adjacent to, or is within a naturally occurring protease recognition site in the unmodified Cry3A toxin.

Specifically exemplified teachings of methods to make modified cry3A nucleic acid molecules that encode modified Cry3A toxins can be found in Example 3. Those skilled in the art will recognize that other methods known in the art can also be used to insert additional protease recognition sites into Cry3A toxins according to the present invention. In another preferred embodiment, the invention encompasses an isolated nucleic acid molecule that encodes a modified Cry3A toxin wherein the additional protease recognition site is inserted in domain I between amino acids corresponding to amino acid numbers 154 and 162 of SEQ ID NO: 2. Preferably, the additional protease recognition site is inserted between amino acid numbers 154 and 162 of SEQ ID NO: 2 or between amino acid numbers 107 and 115 of SEQ ID NO: 4. In a preferred embodiment, the additional protease recognition site is inserted between amino acids corresponding to amino acid numbers 154 and 160 of SEQ ID NO: 2. Preferably, the additional protease recognition site is inserted between amino acid number 154 and 160 of SEQ ID NO: 2 or between amino acid numbers 107 and 113 of SEQ ID NO: 4. Specifically exemplified herein is a nucleic acid molecule, designated cry3A054 (SEQ ID NO: 6), that encodes the modified Cry3A054 toxin (SEQ ID NO: 7) comprising a cathepsin G recognition site inserted in domain I between amino acid numbers 107 and 113 of SEQ ID NO: 4. The cathepsin G recognition site replaces a naturally occurring trypsin recognition site and is adjacent to a naturally occurring chymotrypsin recognition site. When expressed in a heterologous host, the nucleic acid molecule of SEQ ID NO: 6 results in insect control activity against western corn rootworm and northern corn rootworm, showing that the nucleic acid sequence set forth in SEQ ID NO: 6 is sufficient for such insect control activity. In another preferred embodiment, the additional protease recognition site is inserted in domain I between amino acids corresponding to amino acid numbers 154 and 158 of SEQ ID NO: 2. Preferably, the additional protease recognition site is inserted in domain I between amino acid numbers 154 and 158 of SEQ ID NO: 2 or between amino acid numbers 107 and 111 of SEQ ID NO: 4. Specifically exemplified herein are nucleic acid molecules, designated cry3A055 (SEQ ID NO: 8), that encodes the modified Cry3A055 toxin (SEQ ID NO: 9), and cry3A085 (SEQ ID NO: 10), that encodes the modified Cry3A085 toxin (SEQ ID NO: 11), comprising a cathepsin G recognition site inserted in domain I between amino acid numbers 107 and 111 of SEQ ID NO: 4. The cathepsin G recognition site is adjacent to naturally occurring trypsin and chymotrypsin recognition sites. When expressed in a heterologous host, the nucleic acid molecule of SEQ ID NO: 8 or SEQ ID NO: 10 results in insect control activity against western corn rootworm and northern corn rootworm, showing that the nucleic acid sequence set forth in SEQ ID NO: 8 or SEQ ID NO: 10 is sufficient for such insect control activity.

In a preferred embodiment, the invention encompasses an isolated nucleic acid molecule that encodes a modified Cry3A toxin wherein the additional protease recognition site is inserted in domain III between amino acids corresponding to amino acid numbers 583 and 589 of SEQ ID NO: 2. Preferably, the additional protease site is inserted in domain III between amino acid numbers 583 and 589 of SEQ ID NO: 2 or between amino acid numbers 536 and 542 of SEQ ID NO: 4.

In another preferred embodiment, the invention encompasses an isolated nucleic acid molecule that encodes a modified Cry3A toxin wherein the additional protease recognition site is inserted in domain III between amino acids corresponding to amino acid numbers 583 and 588 of SEQ ID NO: 2. Preferably, the additional protease site is inserted in domain III between amino acid numbers 583 and 588 of SEQ ID NO: 2 or between amino acid numbers 536 and 541 of SEQ ID NO: 4. Specifically exemplified herein is a nucleic acid molecule, designated cry3A082 (SEQ ID NO: 12), that encodes the modified Cry3A082 toxin (SEQ ID NO: 13) comprising a cathepsin G recognition site inserted in domain HI between amino acid numbers 536 and 541 of SEQ ID NO: 4. The cathepsin G recognition site replaces a naturally occurring chymotrypsin recognition site. When expressed in a heterologous host, the nucleic acid molecule of SEQ ID NO: 12 results in insect control activity against western corn rootworm and northern corn rootworm, showing that the nucleic acid sequence set forth in SEQ ID NO: 12 is sufficient for such insect control activity.

In another preferred embodiment, the additional protease recognition site is inserted in domain III between amino acids corresponding to amino acid numbers 587 and 588 of SEQ ID NO: 2. Preferably, the additional protease site is inserted in domain III between amino acid numbers 587 and 588 of SEQ ID NO: 2 or between amino acid numbers 540 and 541 of SEQ ID NO: 4. Specifically exemplified herein is a nucleic acid molecule, designated cry3A058 (SEQ ID NO: 14), that encodes the modified Cry3A058 toxin (SEQ ID NO: 15) comprising a cathepsin G recognition site inserted in domain III between amino acid numbers 540 and 541 of SEQ ID NO: 4. The cathepsin G recognition site is within a naturally occurring chymotrypsin recognition site. When expressed in a heterologous host, the nucleic acid molecule of SEQ ID NO: 14 results in insect control activity against western corn rootworm and northern corn rootworm, showing that the nucleic acid sequence set forth in SEQ ID NO: 14 is sufficient for such insect control activity.

In yet another preferred embodiment, the invention encompasses an isolated nucleic acid molecule that encodes a modified Cry3A toxin wherein the additional protease recognition site is inserted in domain I between amino acids corresponding to amino acid numbers 154 and 160 and in domain III between amino acids corresponding to amino acid numbers 587 and 588 of SEQ ID NO: 2. Preferably, the additional protease recognition site is inserted in domain I between amino acid numbers 154 and 160 and in domain III between amino acid numbers 587 and 588 of SEQ ID NO: 2 or in domain I between amino acid numbers 107 and 113 and in domain III between amino acid numbers 540 and 541 of SEQ ID NO: 4.

Specifically exemplified herein is a nucleic acid molecule, designated cry3A057 (SEQ ID NO: 16), that encodes the modified Cry3A057 toxin (SEQ ID NO: 17) comprising a cathepsin G recognition site inserted in domain I between amino acid numbers 107 and 113 and in domain III between amino acid numbers 540 and 541 of SEQ ID NO: 4. The cathepsin G recognition site replaces a naturally occurring trypsin recognition site and is adjacent to a naturally occurring chymotrypsin recognition site in domain I and is within a naturally occurring chymotrypsin recognition site in domain III. When expressed in a heterologous host, the nucleic acid molecule of SEQ ID NO: 16 results in insect control activity against western corn rootworm and northern corn rootworm, showing that the nucleic acid sequence set forth in SEQ ID NO: 16 is sufficient for such insect control activity.

In yet another preferred embodiment, the additional protease recognition site is located in domain I between amino acids corresponding to amino acid numbers 154 and 158 and in domain III between amino acids corresponding to amino acid numbers 587 and 588 of SEQ ID NO: 2. Preferably, the additional protease recognition site is inserted in domain I between amino acid numbers 154 and 158 and in domain III between amino acid numbers 587 and 588 of SEQ ID NO: 2 or in domain I between amino acid numbers 107 and 111 and in domain III between amino acid numbers 540 and 541 of SEQ ID NO: 4.

Specifically exemplified herein is the nucleic acid molecule designated cry3A056 (SEQ ID NO: 18), which encodes the modified Cry3A056 toxin (SEQ ID NO: 19) comprising a cathepsin G recognition site inserted in domain I between amino acid numbers 107 and 111 and in domain III between amino acid numbers 540 and 541 of SEQ ID NO: 4. The cathepsin G recognition site is adjacent to naturally occurring trypsin and chymotrypsin recognition sites in domain I and is within a naturally occurring chymotrypsin recognition site in domain III. When expressed in a heterologous host, the nucleic acid molecule of SEQ ID NO: 18 results in insect control activity against western corn rootworm and northern corn rootworm, showing that the nucleic acid sequence set forth in SEQ ID NO: 18 is sufficient for such insect control activity.

In still another preferred embodiment, the additional protease recognition site is located in domain I between amino acids corresponding to amino acid numbers 154 and 158 and in domain III between amino acids corresponding to amino acid numbers 583 and 588 of SEQ ID NO: 2. Preferably, the additional protease recognition site is inserted in domain I between amino acid numbers 154 and 158 and in domain III between amino acid numbers 583 and 588 of SEQ ID NO: 2 or in domain I between amino acid numbers 107 and 111 and in domain III between amino acid numbers 536 and 541 of SEQ ID NO: 4.

Specifically exemplified herein is a nucleic acid molecule, designated cry3A083 (SEQ ID NO: 20), which encodes the modified Cry3A083 toxin (SEQ ID NO: 21) comprising a cathepsin G recognition site inserted in domain I between amino acid numbers 107 and 111 and in domain III between amino acid numbers 536 and 541 of SEQ ID NO: 4. The cathepsin G recognition site is adjacent to naturally occurring trypsin and chymotrypsin recognition sites in domain I and replaces a naturally occurring chymotrypsin recognition site in domain III. When expressed in a heterologous host, the nucleic acid molecule of SEQ ID NO: 20 results in insect control activity against western corn rootworm and northern corn rootworm, showing that the nucleic acid sequence set forth in SEQ ID NO: 20 is sufficient for such insect control activity.

In a preferred embodiment, the isolated nucleic acid molecule of the present invention comprises nucleotides 1-1791 of SEQ ID NO: 6, nucleotides 1-1806 of SEQ ID NO: 8, nucleotides 1-1812 of SEQ ID NO: 10, nucleotides 1-1794 of SEQ ID NO: 12, nucleotides 1-1818 of SEQ ID NO: 14, nucleotides 1-1812 of SEQ ID NO: 16, nucleotides 1-1791 of SEQ ID NO: 18, and nucleotides 1-1818 of SEQ ID NO: 20.

In another preferred embodiment, the invention encompasses the isolated nucleic acid molecule that encodes a modified Cry3A toxin comprising the amino acid sequence set forth in SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 21.

The present invention also encompasses recombinant vectors comprising the nucleic acid sequences of this invention. In such vectors, the nucleic acid sequences are preferably comprised in expression cassettes comprising regulatory elements for expression of the nucleotide sequences in a host cell capable of expressing the nucleotides sequences. Such regulatory elements usually comprise promoter and termination signals and preferably also comprise elements allowing efficient translation of polypeptides encoded by the nucleic acid sequences of the present invention. Vectors comprising the nucleic acid sequences are usually capable of replication in particular host cells, preferably as extrachromosomal molecules, and are therefore used to amplify the nucleic acid sequences of this invention in the host cells. In one embodiment, host cells for such vectors are microorganisms, such as bacteria, in particular Bacillus thuringiensis or E. coli. In another embodiment, host cells for such recombinant vectors are endophytes or epiphytes. A preferred host cell for such vectors is a eukaryotic cell, such as a plant cell. Plant cells such as maize cells are most preferred host cells. In another preferred embodiment, such vectors are viral vectors and are used for replication of the nucleotide sequences in particular host cells, e.g. insect cells or plant cells. Recombinant vectors are also used for transformation of the nucleotide sequences of this invention into host cells, whereby the nucleotide sequences are stably integrated into the DNA of such host cells. In one, such host cells are prokaryotic cells. In a preferred embodiment, such host cells are eukaryotic cells, such as plant cells. In a most preferred embodiment, the host cells are plant cells, such as maize cells.

In another aspect, the present invention encompasses modified Cry3A toxins produced by the expression of the nucleic acid molecules of the present invention.

In preferred embodiments, the modified Cry3A toxins of the invention comprise a polypeptide encoded by a nucleotide sequence of the invention. In a further preferred embodiment, the modified Cry3A toxin is produced by the expression of the nucleic acid molecule comprising nucleotides 1-1791 of SEQ ID NO: 6, nucleotides 1-1806 of SEQ ID NO: 8, nucleotides 1-1812 of SEQ ID NO: 10, nucleotides 1-1794 of SEQ ID NO: 12, nucleotides 1-1818 of SEQ ID NO: 14, nucleotides 1-1812 of SEQ ID NO: 16, nucleotides 1-1791 of SEQ ID NO: 18, and nucleotides 1-1818 of SEQ ID NO: 20.

In a preferred embodiment, a modified Cry3A toxin of the present invention comprises the amino acid sequence set forth in SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 21.

The modified Cry3A toxins of the present invention have insect control activity when tested against insect pests in bioassays. In another preferred embodiment, the modified Cry3A toxins of the invention are active against coleopteran insects, preferably against western corn rootworm and northern corn rootworm. The insect controlling properties of the modified Cry3A toxins of the invention are further illustrated in Examples 4 and 6.

The present invention also encompasses a composition comprising an effective insect-controlling amount of a modified Cry3A toxin according to the invention. In another preferred embodiment, the invention encompasses a method of producing a modified Cry3A toxin that is active against insects, comprising: (a) obtaining a host cell comprising a chimeric gene, which itself comprises a heterologous promoter sequence operatively linked to the nucleic acid molecule of the invention; and (b) expressing the nucleic acid molecule in the transgenic host cell, which results in at least one modified Cry3A toxin that is active against insects.

In a further preferred embodiment, the invention encompasses a method of producing an insect-resistant transgenic plant, comprising introducing a nucleic acid molecule of the invention into the transgenic plant, wherein the nucleic acid molecule is expressible in the transgenic plant in an effective amount to control insects. In a preferred embodiment, the insects are coleopteran insects, preferably western corn rootworm and northern corn rootworm.

In yet a further preferred embodiment, the invention encompasses a method of controlling insects, comprising delivering to the insects an effective amount of a modified Cry3A toxin of the invention. According to this embodiment, the insects are coleopteran insects, preferably, western corn rootworm and northern corn rootworm. Preferably, the modified Cry3A toxin is delivered to the insects orally. In one preferred aspect, the toxin is delivered orally through a transgenic plant comprising a nucleic acid sequence that expresses a modified Cry3A toxin of the present invention.

The present invention also encompasses a method of making a modified Cry3A toxin, comprising: (a) obtaining a cry3A toxin gene which encodes a Cry3A toxin; (b) identifying a gut protease of a target insect; (c) obtaining a nucleotide sequence which encodes a recognition site for the gut protease; (d) inserting the nucleotide sequence of (c) into either domain I or domain III or both domain I and domain III at a position that replaces, is within, or adjacent to a nucleotide sequence that codes for a naturally occurring protease recognition site in the cry3A toxin gene, thus creating a modified cry3A toxin gene; (e) inserting the modified cry3A toxin gene in an expression cassette; (f) expressing the modified cry3A toxin gene in a non-human host cell, resulting in the host cell producing a modified Cry3A toxin; and, (g) bioassaying the modified Cry3A toxin against a target insect, which causes higher mortality to the target insect than the mortality caused by a Cry3A toxin. In a preferred embodiment, the modified Cry3A toxin causes at least about 50% mortality to the target insect when the Cry3A toxin causes up to about 30% mortality.

The present invention further encompasses a method of controlling insects wherein the transgenic plant further comprises a second nucleic acid sequence or groups of nucleic acid sequences that encode a second pesticidal principle. Particularly preferred second nucleic acid sequences are those that encode a δ-endotoxin, those that encode a Vegetative Insecticidal Protein toxin, disclosed in U.S. Pat. Nos. 5,849,870 and 5,877,012, incorporated herein by reference, or those that encode a pathway for the production of a non-proteinaceous principle.

In further embodiments, the nucleotide sequences of the invention can be further modified by incorporation of random mutations in a technique known as in vitro recombination or DNA shuffling. This technique is described in Stemmer et al., Nature 370:389-391 (1994) and U.S. Pat. No. 5,605,793, which are incorporated herein by reference. Millions of mutant copies of a nucleotide sequence are produced based on an original nucleotide sequence of this invention and variants with improved properties, such as increased insecticidal activity, enhanced stability, or different specificity or ranges of target-insect pests are recovered. The method encompasses forming a mutagenized double-stranded polynucleotide from a template double-stranded polynucleotide comprising a nucleotide sequence of this invention, wherein the template double-stranded polynucleotide has been cleaved into double-stranded-random fragments of a desired size, and comprises the steps of adding to the resultant population of double-stranded random fragments one or more single or double-stranded oligonucleotides, wherein said oligonucleotides comprise an area of identity and an area of heterology to the double-stranded template polynucleotide; denaturing the resultant mixture of double-stranded random fragments and oligonucleotides into single-stranded fragments; incubating the resultant population of single-stranded fragments with a polymerase under conditions which result in the annealing of said single-stranded fragments at said areas of identity to form pairs of annealed fragments, said areas of identity being sufficient for one member of a pair to prime replication of the other, thereby forming a mutagenized double-stranded polynucleotide; and repeating the second and third steps for at least two further cycles, wherein the resultant mixture in the second step of a further cycle includes the mutagenized double-stranded polynucleotide from the third step of the previous cycle, and the further cycle forms a further mutagenized double-stranded polynucleotide. In a preferred embodiment, the concentration of a single species of double-stranded random fragment in the population of double-stranded random fragments is less than 1% by weight of the total DNA. In a further preferred embodiment, the template double-stranded polynucleotide comprises at least about 100 species of polynucleotides. In another preferred embodiment, the size of the double-stranded random fragments is from about 5 bp to 5 kb. In a further preferred embodiment, the fourth step of the method comprises repeating the second and the third steps for at least 10 cycles.

Expression of the Nucleotide Sequences in Heterologous Microbial Hosts

As biological insect control agents, the insecticidal modified Cry3A toxins are produced by expression of the nucleotide sequences in heterologous host cells capable of expressing the nucleotide sequences. In a first embodiment, B. thuringiensis cells comprising modifications of a nucleotide sequence of this invention are made. Such modifications encompass mutations or deletions of existing regulatory elements, thus leading to altered expression of the nucleotide sequence, or the incorporation of new regulatory elements controlling the expression of the nucleotide sequence. In another embodiment, additional copies of one or more of the nucleotide sequences are added to Bacillus thuringiensis cells either by insertion into the chromosome or by introduction of extrachromosomally replicating molecules containing the nucleotide sequences.

In another embodiment, at least one of the nucleotide sequences of the invention is inserted into an appropriate expression cassette, comprising a promoter and termination signal. Expression of the nucleotide sequence is constitutive, or an inducible promoter responding to various types of stimuli to initiate transcription is used. In a preferred embodiment, the cell in which the toxin is expressed is a microorganism, such as a virus, bacteria, or a fungus. In a preferred embodiment, a virus, such as a baculovirus, contains a nucleotide sequence of the invention in its genome and expresses large amounts of the corresponding insecticidal toxin after infection of appropriate eukaryotic cells that are suitable for virus replication and expression of the nucleotide sequence. The insecticidal toxin thus produced is used as an insecticidal agent. Alternatively, baculoviruses engineered to include the nucleotide sequence are used to infect insects in vivo and kill them either by expression of the insecticidal toxin or by a combination of viral infection and expression of the insecticidal toxin.

Bacterial cells are also hosts for the expression of the nucleotide sequences of the invention. In a preferred embodiment, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive nucleotide sequences for the same purpose.

Techniques for these genetic manipulations are specific for the different available hosts and are known in the art. For example, the expression vectors pKK223-3 and pKK223-2 can be used to express heterologous genes in E. coli, either in transcriptional or translational fusion, behind the tac or trc promoter. For the expression of operons encoding multiple ORFs, the simplest procedure is to insert the operon into a vector such as pKK223-3 in transcriptional fusion, allowing the cognate ribosome binding site of the heterologous genes to be used. Techniques for overexpression in gram-positive species such as Bacillus are also known in the art and can be used in the context of this invention (Quax et al. In:Industrial Microorganisms:Basic and Applied Molecular Genetics, Eds. Baltz et al., American Society for Microbiology, Washington (1993)). Alternate systems for overexpression rely for example, on yeast vectors and include the use of Pichia, Saccharomyces and Kluyveromyces (Sreekrishna, In:Industrial microorganisms:basic and applied molecular genetics, Baltz, Hegeman, and Skatrud eds., American Society for Microbiology, Washington (1993); Dequin & Barre, Biotechnology L2:173-177 (1994); van den Berg et al., Biotechnology 8:135-139 (1990)).

Plant Transformation

In a particularly preferred embodiment, at least one of the insecticidal modified Cry3A toxins of the invention is expressed in a higher organism, e.g., a plant. In this case, transgenic plants expressing effective amounts of the modified Cry3A toxins protect themselves from insect pests. When the insect starts feeding on such a transgenic plant, it also ingests the expressed modified Cry3A toxins. This will deter the insect from further biting into the plant tissue or may even harm or kill the insect. A nucleotide sequence of the present invention is inserted into an expression cassette, which is then preferably stably integrated in the genome of said plant. In another preferred embodiment, the nucleotide sequence is included in a non-pathogenic self-replicating virus. Plants transformed in accordance with the present invention may be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees.

Once a desired nucleotide sequence has been transformed into a particular plant species, it may be propagated in that species or moved into other varieties of the same species, particularly including commercial varieties, using traditional breeding techniques. A nucleotide sequence of this invention is preferably expressed in transgenic plants, thus causing the biosynthesis of the corresponding modified Cry3A toxin in the transgenic plants. In this way, transgenic plants with enhanced resistance to insects are generated. For their expression in transgenic plants, the nucleotide sequences of the invention may require other modifications and optimization. Although in many cases genes from microbial organisms can be expressed in plants at high levels without modification, low expression in transgenic plants may result from microbial nucleotide sequences having codons that are not preferred in plants. It is known in the art that all organisms have specific preferences for codon usage, and the codons of the nucleotide sequences described in this invention can be changed to conform with plant preferences, while maintaining the amino acids encoded thereby. Furthermore, high expression in plants is best achieved from coding sequences that have at least about 35% GC content, preferably more than about 45%, more preferably more than about 50%, and most preferably more than about 60%. Microbial nucleotide sequences that have low GC contents may express poorly in plants due to the existence of ATTTA motifs that may destabilize messages, and AATAAA motifs that may cause inappropriate polyadenylation. Although preferred gene sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). In addition, the nucleotide sequences are screened for the existence of illegitimate splice sites that may cause message truncation. All changes required to be made within the nucleotide sequences such as those described above are made using well known techniques of site directed mutagenesis, PCR, and synthetic gene construction using the methods described in the published patent applications EP 0 385 962 (to Monsanto), EP 0 359 472 (to Lubrizol, and WO 93/07278 (to Ciba-Geigy).

In one embodiment of the invention a cry3A gene is made according to the procedure disclosed in U.S. Pat. No. 5,625,136, herein incorporated by reference. In this procedure, maize preferred codons, i.e., the single codon that most frequently encodes that amino acid in maize, are used. The maize preferred codon for a particular amino acid might be derived, for example, from known gene sequences from maize. Maize codon usage for 28 genes from maize plants is found in Murray et al., Nucleic Acids Research 17:477-498 (1989), the disclosure of which is incorporated herein by reference. A synthetic sequence made with maize optimized codons is set forth in SEQ ID NO: 3.

In this manner, the nucleotide sequences can be optimized for expression in any plant. It is recognized that all or any part of the gene sequence may be optimized or synthetic. That is, synthetic or partially optimized sequences may also be used.

For efficient initiation of translation, sequences adjacent to the initiating methionine may require modification. For example, they can be modified by the inclusion of sequences known to be effective in plants. Joshi has suggested an appropriate consensus for plants (NAR 15:6643-6653 (1987)) and Clonetech suggests a further consensus translation initiator (1993/1994 catalog, page 210). These consensuses are suitable for use with the nucleotide sequences of this invention. The sequences are incorporated into constructions comprising the nucleotide sequences, up to and including the ATG (whilst leaving the second amino acid unmodified), or alternatively up to and including the GTC subsequent to the ATG (with the possibility of modifying the second amino acid of the transgene). Expression of the nucleotide sequences in transgenic plants is driven by promoters that function in plants. The choice of promoter will vary depending on the temporal and spatial requirements for expression, and also depending on the target species. Thus, expression of the nucleotide sequences of this invention in leaves, in stalks or stems, in ears, in inflorescences (e.g. spikes, panicles, cobs, etc.), in roots, and/or seedlings is preferred. In many cases, however, protection against more than one type of insect pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

Preferred promoters that are expressed constitutively include promoters from genes encoding actin or ubiquitin and the CaMV 35S and 19S promoters. The nucleotide sequences of this invention can also be expressed under the regulation of promoters that are chemically regulated. This enables the insecticidal modified Cry3A toxins to be synthesized only when the crop plants are treated with the inducing chemicals. Preferred technology for chemical induction of gene expression is detailed in the published application EP 0 332 104 (to Ciba-Geigy) and U.S. Pat. No. 5,614,395. A preferred promoter for chemical induction is the tobacco PR-1a promoter.

A preferred category of promoters is that which is wound inducible. Numerous promoters have been described which are expressed at wound sites and also at the sites of phytopathogen infection. Ideally, such a promoter should only be active locally at the sites of infection, and in this way the insecticidal modified Cry3A toxins only accumulate in cells that need to synthesize the insecticidal modified Cry3A toxins to kill the invading insect pest. Preferred promoters of this kind include those described by Stanford et al. Mol. Gen. Genet. 215:200-208 (1989), Xu et al. Plant Molec. Biol. 22:573-588 (1993), Logemann et al. Plant Cell 1:151-158 (1989), Rohrmeier & Lehle, Plant Molec. Biol. 22:783-792 (1993), Firek et al. Plant Molec. Biol. 22:129-142 (1993), and Warner et al. Plant J. 3:191-201 (1993).

Tissue-specific or tissue-preferential promoters useful for the expression of the modified Cry3A toxin genes in plants, particularly maize, are those which direct expression in root, pith, leaf or pollen, particularly root. Such promoters, e.g. those isolated from PEPC or trpA, are disclosed in U.S. Pat. No. 5,625,136, or MTL, disclosed in U.S. Pat. No. 5,466,785. Both U.S. patents are herein incorporated by reference in their entirety. Further preferred embodiments are transgenic plants expressing the nucleotide sequences in a wound-inducible or pathogen infection-inducible manner.

In addition to promoters, a variety of transcriptional terminators are also available for use in chimeric gene construction using the modified Cry3A toxin genes of the present invention. Transcriptional terminators are responsible for the termination of transcription beyond the transgene and its correct polyadenylation. Appropriate transcriptional terminators and those that are known to function in plants include the CaMV 35S terminator, the tml terminator, the nopaline synthase terminator, the pea rbcS E9 terminator and others known in the art. These can be used in both monocotyledons and dicotyledons. Any available terminator known to function in plants can be used in the context of this invention.

Numerous other sequences can be incorporated into expression cassettes described in this invention. These include sequences that have been shown to enhance expression such as intron sequences (e.g. from Adhl and bronzel) and viral leader sequences (e.g. from TMV, MCMV and AMV).

It may be preferable to target expression of the nucleotide sequences of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, the DNA encoding the target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well known in the art. Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors comprising the modified Cry3A toxin genes of the present invention may also comprise genes (e.g. phosphomannose isomerase; PMI) which provide for positive selection of the transgenic plants as disclosed in U.S. Pat. Nos. 5,767,378 and 5,994,629, herein incorporated by reference. The choice of selectable marker is not, however, critical to the invention.

In another embodiment, a nucleotide sequence of the present invention is directly transformed into the plastid genome. A major advantage of plastid transformation is that plastids are generally capable of expressing bacterial genes without substantial codon optimization, and plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransf erase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

Combinations of Insect Control Principles

The modified Cry3A toxins of the invention can be used in combination with Bt δ-endotoxins or other pesticidal principles to increase pest target range. Furthermore, the use of the modified Cry3A toxins of the invention in combination with Bt δ-endotoxins or other pesticidal principles of a distinct nature has particular utility for the prevention and/or management of insect resistance.

Other insecticidal principles include, for example, lectins, α-amylase, peroxidase and cholesterol oxidase. Vegetative Insecticidal Protein genes, such as vip1A(a) and vip2A(a) as disclosed in U.S. Pat. No. 5,889,174 and herein incorporated by reference, are also useful in the present invention.

This co-expression of more than one insecticidal principle in the same transgenic plant can be achieved by genetically engineering a plant to contain and express all the genes necessary. Alternatively, a plant, Parent 1, can be genetically engineered for the expression of genes of the present invention. A second plant, Parent 2, can be genetically engineered for the expression of a supplemental insect control principle. By crossing Parent 1 with Parent 2, progeny plants are obtained which express all the genes introduced into Parents 1 and 2.

Transgenic seed of the present invention can also be treated with an insecticidal seed coating as described in U.S. Pat. Nos. 5,849,320 and 5,876,739, herein incorporated by reference. Where both the insecticidal seed coating and the transgenic seed of the invention are active against the same target insect, the combination is useful (i) in a method for enhancing activity of a modified Cry3A toxin of the invention against the target insect and (ii) in a method for preventing development of resistance to a modified Cry3A toxin of the invention by providing a second mechanism of action against the target insect. Thus, the invention provides a method of enhancing activity against or preventing development of resistance in a target insect, for example corn rootworm, comprising applying an insecticidal seed coating to a transgenic seed comprising one or more modified Cry3A toxins of the invention.

Even where the insecticidal seed coating is active against a different insect, the insecticidal seed coating is useful to expand the range of insect control, for example by adding an insecticidal seed coating that has activity against lepidopteran insects to the transgenic seed of the invention, which has activity against coleopteran insects, the coated transgenic seed produced controls both lepidopteran and coleopteran insect pests.

### EXAMPLES

The invention will be further described by reference to the following detailed examples. These examples are provided for the purposes of illustration only, and are not intended to be limiting unless otherwise specified. Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1

### Maize Optimized cry3A Gene Construction

The maize optimized cry3A gene is made according to the procedure disclosed in U.S. Pat. No. 5,625,136. In this procedure, maize preferred codons, i.e., the single codon that most frequently encodes that amino acid in maize, are used. The maize preferred codon for a particular amino acid is derived from known gene sequences from maize. Maize codon usage for 28 genes from maize plants is found in Murray et al., Nucleic Acids Research 17:477-498 (1989). The synthetic sequence made with maize optimized codons is set forth in SEQ ID NO: 3.

### Example 2

### Identification of Cathepsin-G Enzymatic Activity in Western Corn Rootworm Guts

Cathepsin G-like (serine protease) and cathepsin B-like (cysteine protease) enzymatic activities in western corn rootworm guts are measured using colorimetric substrates. Each 1 ml reaction contains five homogenized midguts of the 3rd instar of western corn rootworm and 1 mg of substrate dissolved in reaction buffer (10 mM Tris, 5 mM NaCl, 0.01 M DTT, pH 7.5). The cathepsin G substrate tested is Ala-Ala-Pro-Phe (SEQ ID NO: 35)-pNA and cathepsin B substrate, Arg-Arg-pNA. The reactions are incubated at 28° C. for 1 hr. The intensity of yellow color formation, indicative of the efficiency of a protease to recognize the appropriate substrate, is compared in treatments vs. controls. The reactions are scored as negative (−) if no color or slight background color is detected. Reactions which are 25%, 50%, 75% or 100% above background are scored as +, ++, +++, or ++++, respectively.

Results of the enzymatic assays are shown in the following table.

TABLE 1

| Reaction | Product Color intensity |
|---|---|
| WCR gut only | − |
| Cathepsin B substrate only | − |
| Cathepsin G substrate only | − |
| WCR gut + Cathepsin B substrate | + |
| WCR gut + Cathepsin G substrate | +++ |

This is the first time that the serine protease cathepsin G activity has been identified in western corn rootworm guts. Western corn rootworm guts clearly have stronger cathepsin G, the serine protease, activity compared to cathepsin B, the cysteine protease, activity. The AAPF sequence (SEQ ID NO: 35) is selected as the cathepsin G protease recognition site for creating modified Cry3A toxins of the present invention.

### Example 3

### Construction of Modified cry3A Genes

Modified cry3A genes comprising a nucleotide sequence that encodes the cathepsin G recognition site in domain I, domain III, or domain I and domain III are made using overlap PCR. The maize optimized cry3A gene (SEQ ID NO: 2), comprised in plasmid pCIB6850 (SEQ ID NO: 5), is used as the starting template. Eight modified cry3A gene constructs, which encode modified Cry3A toxins, are made; cry3A054, cry3A055, and cry3A085, which comprise the cathepsin G recognition site coding sequence in domain I; cry3A058, cry3A082, which comprise the cathepsin G recognition site coding sequence in domain III; cry3A056, cry3A057, cry3A083, which comprise the cathepsin G recognition site coding sequence in domain I and domain III. The eight modified cry3A genes and the modified Cry3A toxins they encode are described as follows:

cry3A054 comprised in pCMS054

cry3A054 (SEQ ID NO: 6) comprises a nucleotide sequence encoding a modified Cry3A toxin. Three overlap PCR primer pairs are used to insert the nucleotide sequence encoding the cathepsin G recognition site into the unmodified maize optimized cry3A:

(SEQ ID NO: 22)

| | | |
|---|---|---|
| 1. | BamExt1- | 5′-GGATCCACCATGACGGCCGAC-3′ |

(SEQ ID NO: 23)

| | |
|---|---|
| AAPFtail3- | 5′-GAACGGTGCAGCGGGGTTCTTCTGCCAGC-3′ |

(SEQ ID NO: 24)

| | | |
|---|---|---|
| 2. | Tail5mod- | 5′-GCTGCACCGTTCCCCCACAGCCAGGGCCG-3′ |

(SEQ ID NO: 25)

| | |
|---|---|
| XbaIExt2- | 5′-TCTAGACCCACGTTGTACCAC-3′ |

(SEQ ID NO: 22)

| | | |
|---|---|---|
| 3. | BamExt1- | 5′-GGATCCACCATGACGGCCGAC-3′ |

(SEQ ID NO: 25)

| | |
|---|---|
| XbaIExt2- | 5′-TCTAGACCCACGTTGTACCAC-3′ |

Primer pair 1 and primer pair 2 generate two unique PCR products. These products are then combined in equal parts and primer pair 3 is used to join the products to generate one PCR fragment that is cloned back into the original pCIB6850 template. The modified cry3A054 gene is then transferred to pBluescript (Stratagene). The resulting plasmid is designated pCMS054 and comprises the cry3A054 gene (SEQ ID NO: 6).

The modified Cry3A054 toxin (SEQ ID NO: 7), encoded by the modified cry3A gene comprised in pCMS054, has a cathepsin G recognition site, comprising the amino acid sequence AAPF (SEQ ID NO: 35), inserted in domain I between amino acids 107 and 113 of the unmodified Cry3A toxin (SEQ ID NO: 4). The cathepsin G recognition site replaces the naturally occurring trypsin recognition site and is adjacent to a naturally occurring chymotrypsin recognition site.

cry3A055 Comprised in pCMS055

cry3A055 (SEQ ID NO: 8) comprises a nucleotide sequence encoding a modified Cry3A toxin. Three overlap PCR primer pairs are used to insert the nucleotide sequence encoding the cathepsin G recognition site into the unmodified maize optimized cry3A:

(SEQ ID NO: 22)

| | | |
|---|---|---|
| 1. | BamExt1- | 5′-GGATCCACCATGACGGCCGAC-3′ |

(SEQ ID NO: 23)

| | |
|---|---|
| AAPFtail3- | 5′-GAACGGTGCAGCGGGGTTCTTCTGCCAGC-3′ |

(SEQ ID NO: 26)

| | | |
|---|---|---|
| 2. | AAPFtail4- | 5′-GCTGCACCGTTCCGCAACCCCCACAGCCA-3′ |

(SEQ ID NO: 25)

| | |
|---|---|
| XbaIExt2- | 5′-TCTAGACCCACGTTGTACCAC-3′ |

(SEQ ID NO: 22)

| | | |
|---|---|---|
| 3. | BamExt1- | 5′-GGATCCACCATGACGGCCGAC-3′ |

(SEQ ID NO: 25)

| | |
|---|---|
| XbaIExt2- | 5′-TCTAGACCCACGTTGTACCAC-3′ |

Primer pair 1 and primer pair 2 generate two unique PCR products. These products are then combined in equal parts and primer pair 3 is used to join the products to generate one PCR fragment that is cloned back into the original pCIB6850 template. The modified cry3A055 gene is then transferred to pBluescript (Stratagene); The resulting plasmid is designated pCMS055 and comprises the cry3A055 gene (SEQ ID NO: 8).

The modified Cry3A055 toxin (SEQ ID NO: 9), encoded by the modified cry3A gene comprised in pCMS055, has a cathepsin G recognition site, comprising the amino acid sequence AAPF (SEQ ID NO: 35), inserted in domain I between amino acids 107 and 111 of the unmodified Cry3A toxin (SEQ ID NO: 4). The cathepsin G recognition site is adjacent to a natural trypsin and chymotrypsin recognition site.

cry3A058 Comprised in pCMS058

cry3A058 (SEQ ID NO: 14) comprises a nucleotide sequence encoding a modified Cry3A toxin. Three overlap PCR primer pairs are used to insert the nucleotide sequence encoding the cathepsin G recognition site into the unmodified maize optimized cry3A:

(SEQ ID NO: 27)

| | | |
|---|---|---|
| 1. | SalExt- | 5′-GAGCGTCGACTTCTTCAAC-3′ |

(SEQ ID NO: 28)

| | |
|---|---|
| AAPF-Y2- | 5′-GAACGGTGCAGCGTATTGGTTGAAGGGGGC-3′ |

(SEQ ID NO: 29)

| | | |
|---|---|---|
| 2. | AAPF-Y1- | 5′-GCTGCACCGTTCTACTTCGACAAGACCATC-3′ |

(SEQ ID NO: 30)

| | |
|---|---|
| SacExt- | 5′-GAGCTCAGATCTAGTTCACGG-3′ |

(SEQ ID NO: 27)

| | | |
|---|---|---|
| 3. | SalExt- | 5′-GAGCGTCGACTTCTTCAAC-3′ |

(SEQ ID NO: 30)

| | |
|---|---|
| SacExt- | 5′-GAGCTCAGATCTAGTTCACGG-3′ |

Primer pair 1 and primer pair 2 generate two unique PCR products. These products are then combined in equal parts and primer pair 3 is used to join the products to generate one PCR fragment that is cloned back into the original pCIB6850 template. The modified cry3A058 gene is then transferred to pBluescript (Stratagene). The resulting plasmid is designated pCMS058 and comprises the cry3A058 gene (SEQ ID NO: 14).

The modified Cry3A058 toxin (SEQ ID NO: 15), encoded by the modified cry3A gene, has a cathepsin G recognition site, comprising the amino acid sequence AAPF (SEQ ID NO: 35), inserted in domain III between amino acids 540 and 541 of the unmodified Cry3A toxin (SEQ ID NO: 4). The cathepsin G recognition site is within a naturally occurring chymotrypsin recognition site.

pCMS082 Comprising cry3A082

cry3A082 (SEQ ID NO: 12) comprises a nucleotide sequence encoding a modified Cry3A toxin. A QuikChange Site Directed Mutagenesis PCR primer pair is used to insert the nucleotide sequence encoding the cathepsin G recognition site into the unmodified maize optimized cry3A:

BBmod1-5′-CGGGGCCCCCGCTGCACCGTTCTACTTCGACA-3′ (SEQ ID NO: 31)

BBmod2-5′-TGTCGAAGTAGAACGGTGCAGCGGGGGCCCCG-3′ (SEQ ID NO: 32)

The primer pair generates a unique PCR product. This product is cloned back into the original pCIB6850 template. The modified cry3A082 gene is then transferred to pBluescript (Stratagene). The resulting plasmid is designated pCMS082 and comprises the cry3A082 gene (SEQ ID NO: 12).

The modified Cry3A082 toxin (SEQ ID NO: 13), encoded by the modified cry3A gene, has a cathepsin G recognition site, comprising the amino acid sequence AAPF (SEQ ID NO: 35), inserted in domain Ill between amino acids 539 and 542 of the unmodified Cry3A toxin (SEQ ID NO: 4). The cathepsin G recognition site replaces a naturally occurring chymotrypsin recognition site.

cry3A056 Comprised in pCMS056

cry3A056 (SEQ ID NO: 18) comprises a nucleotide sequence encoding a modified Cry3A toxin. Six overlap PCR primer pairs are used to insert two cathepsin G recognition sites into the unmodified cry3A:

(SEQ ID NO: 22)

| | | |
|---|---|---|
| 1. | BamExt1- | 5′-GGATCCACCATGACGGCCGAC-3′ |

(SEQ ID NO: 23)

| | |
|---|---|
| AAPFtail3- | 5′-GAACGGTGCAGCGGGGTTCTTCTGCCAGC-3′ |

(SEQ ID NO: 26)

| | | |
|---|---|---|
| 2. | AAPFtail4- | 5′-GCTGCACCGTTCCGCAACCCCCACAGCCA-3′ |

(SEQ ID NO: 25)

| | |
|---|---|
| XbaIExt2- | 5′-TCTAGACCCACGTTGTACCAC-3′ |

(SEQ ID NO: 22)

| | | |
|---|---|---|
| 3. | BamExt1- | 5′-GGATCCACCATGACGGCCGAC-3′ |

(SEQ ID NO: 25)

| | |
|---|---|
| XbaIExt2- | 5′-TCTAGACCCACGTTGTACCAC-3′ |

(SEQ ID NO: 27)

| | | |
|---|---|---|
| 4. | SalExt- | 5′-GAGCGTCGACTTCTTCAAC-3′ |

(SEQ ID NO: 28)

| | |
|---|---|
| AAPF-Y2- | 5′-GAACGGTGCAGCGTATTGGTTGAAGGGGGC-3′ |

(SEQ ID NO: 29)

| | | |
|---|---|---|
| 5. | AAPF-Y1- | 5′-GCTGCACCGTTCTACTTCGACAAGACCATC-3′ |

(SEQ ID NO: 30)

| | |
|---|---|
| SacExt- | 5′-GAGCTCAGATCTAGTTCACGG-3′ |

(SEQ ID NO: 27)

| | | |
|---|---|---|
| 6. | SalExt- | 5′-GAGCGTCGACTTCTTCAAC-3′ |

(SEQ ID NO: 30)

| | |
|---|---|
| SacExt- | 5′-GAGCTCAGATCTAGTTCACGG-3′ |

Primer pair 1 and primer pair 2 generate two unique PCR products. These products are combined in equal parts and primer pair 3 is used to join the products to generate one PCR fragment that is cloned back into the original pCIB6850 plasmid. The modified cry3A055 gene is then transferred to pBluescript (Stratagene). The resulting plasmid is designated pCMS055. Primer pair 4 and primer pair 5 generate another unique set of fragments that are joined by another PCR with primer pair 6. This fragment is cloned into domain III of the modified cry3A055 gene comprised in pCMS055. The resulting plasmid is designated pCMS056 and comprises the cry3A056 gene (SEQ ID NO: 18).

The modified Cry3A056 toxin (SEQ ID NO: 19), encoded by the modified cry3A gene, has a cathepsin G recognition site, comprising the amino acid sequence AAPF (SEQ ID NO: 35), inserted in domain I between amino acids 107 and 111 and in domain III between amino acids 540 and 541 of the unmodified Cry3A toxin (SEQ ID NO: 4). The cathepsin G recognition site is adjacent to a naturally occurring trypsin and chymotrypsin recognition site in domain I and is within a naturally occurring chymotrypsin recognition site in domain III.

cry3A057 Comprised in pCMS057

cry3A057 (SEQ ID NO: 16) comprises a nucleotide sequence encoding a modified Cry3A toxin. Six overlap PCR primer pairs are used to insert two cathepsin G recognition sites into the unmodified cry3A:

(SEQ ID NO: 22)

| | | |
|---|---|---|
| 1. | BamExt1- | 5′-GGATCCACCATGACGGCCGAC-3′ |

(SEQ ID NO: 23)

| | |
|---|---|
| AAPFtail3- | 5′-GAACGGTGCAGCGGGGTTCTTCTGCCAGC-3′ |

(SEQ ID NO: 24)

| | | |
|---|---|---|
| 2. | Tail5mod- | 5′-GCTGCACCGTTCCCCCACAGCCAGGGCCG-3′ |

(SEQ ID NO: 25)

| | |
|---|---|
| XbaIExt2- | 5′-TCTAGACCCACGTTGTACCAC-3′ |

(SEQ ID NO: 22)

| | | |
|---|---|---|
| 3. | BamExt1- | 5′-GGATCCACCATGACGGCCGAC-3′ |

(SEQ ID NO: 25)

| | |
|---|---|
| XbaIExt2- | 5′-TCTAGACCCACGTTGTACCAC-3′ |

(SEQ ID NO: 27)

| | | |
|---|---|---|
| 4. | SalExt- | 5′-GAGCGTCGACTTCTTCAAC-3′ |

(SEQ ID NO: 28)

| | |
|---|---|
| AAPF-Y2- | 5′-GAACGGTGCAGCGTATTGGTTGAAGGGGGC-3′ |

(SEQ ID NO: 29)

| | | |
|---|---|---|
| 5. | AAPF-Y1- | 5′-GCTGCACCGTTCTACTTCGACAAGACCATC-3′ |

(SEQ ID NO: 30)

| | |
|---|---|
| SacExt- | 5′-GAGCTCAGATCTAGTTCACGG-3′ |

(SEQ ID NO: 27)

| | | |
|---|---|---|
| 6. | SalExt- | 5′-GAGCGTCGACTTCTTCAAC-3′ |

(SEQ ID NO: 30)

| | |
|---|---|
| SacExt- | 5′-GAGCTCAGATCTAGTTCACGG-3′ |

Primer pair 1 and primer pair 2 generate two unique PCR products. These products are combined in equal parts and primer pair 3 is used to join the products to generate one PCR fragment that is cloned back into the original pCIB6850 plasmid. The modified cry3A054 gene is then transferred to pBluescript (Stratagene). The resulting plasmid is designated pCMS054. Primer pair 4 and primer pair 5 generate another unique set of fragments that are joined by another PCR with primer pair 6. This fragment is cloned into domain III of the modified cry3A054 gene comprised in pCMS054. The resulting plasmid is designated pCMS057 and comprises the cry3A057 gene (SEQ ID NO: 16).

The modified Cry3A057 toxin (SEQ ID NO: 17), encoded by the modified cry3A gene, has a cathepsin G recognition site, comprising the amino acid sequence AAPF (SEQ ID NO: 35), inserted in domain I between amino acids 107 and 113 and in domain III between amino acids 540 and 541 of the unmodified Cry3A toxin (SEQ ID NO: 4). The cathepsin G recognition site replaces a naturally occurring trypsin recognition site and is adjacent to a naturally occurring chymotrypsin recognition site in domain I and is within a naturally occurring chymotrypsin recognition site in domain III.

cry3A083 Comprised in pCMS083

cry3A083 (SEQ ID NO: 20) comprises a nucleotide sequence encoding a modified Cry3A toxin. Three overlap PCR primer pairs and one QuikChange Site Directed Mutagenesis PCR primer pair are used to insert two cathepsin G recognition sites into the unmodified cry3A:

(SEQ ID NO: 22)

| | |
|---|---|
| 1. | |
| BamExt1- | 5′-GGATCCACCATGACGGCCGAC-3′ |

(SEQ ID NO: 23)

| | |
|---|---|
| AAPFtail3- | 5′-GAACGGTGCAGCGGGGTTCTTCTGCCAGC-3′ |

(SEQ ID NO: 26)

| | |
|---|---|
| 2. | |
| AAPFtail4- | 5′-GCTGCAGCGTTCCGCAACCCCCACAGCCA-3′ |

(SEQ ID NO: 25)

| | |
|---|---|
| XbaIExt2- | 5′-TCTAGACCCACGTTGTACCAC-3′ |

(SEQ ID NO: 22)

| | |
|---|---|
| 3. | |
| BamExt1- | 5′-GGATCCACCATGACGGCCGAC-3′ |

(SEQ ID NO: 25)

| | |
|---|---|
| XbaIExt2- | 5′-TCTAGACCCACGTTGTACCAC-3′ |

(SEQ ID NO: 31)

| | |
|---|---|
| BBmod1- | 5′-CGGGGCCCCCGCTGCACCGTTCTACTTCGACA-3 |

(SEQ ID NO: 32)

| | |
|---|---|
| BBmod2- | 5′-TGTCGAAGTAGAACGGTGCAGCGGGGGCCCCG-3′ |

Primer pair 1 and primer pair 2 generate two unique PCR products. These products are combined in equal parts and primer pair 3 is used to join the products to generate one PCR fragment that is cloned back into the original pCIB6850 plasmid. The modified cry3A055 gene is then transferred to pBluescript (Stratagene). The resulting plasmid is designated pCMS055. Primer pair 4 generates another unique fragment that is cloned into domain III of the modified cry3A comprised in pCMS055. The resulting plasmid is designated pCMS083 and comprises the cry3A083 gene (SEQ ID NO: 20).

The modified Cry3A083 toxin (SEQ ID NO: 21), encoded by the modified cry3A gene, has a cathepsin G recognition site, comprising the amino acid sequence AAPF (SEQ ID NO: 35), inserted in domain I between amino acids 107 and 111 and between amino acids 539 and 542 of the unmodified Cry3A toxin (SEQ ID NO: 4). The cathepsin G recognition site is adjacent to a naturally occurring trypsin and chymotrypsin recognition site in domain I and replaces a naturally occurring chymotrypsin recognition site in domain III.

cry3A085 Comprised in pCMS085

The cry3A085 gene (SEQ ID NO: 10) comprises a cathepsin G coding sequence at the same position as in the cry3A055 gene described above. The cry3A085 gene has an additional 24 nucleotides inserted at the 5′ end which encode amino acids 41-47 of the deduced amino acid sequence set forth in SEQ ID NO: 2 as well as an additional methionine. The additional nucleotides are inserted at the 5′ end of the cry3A055 gene using the following PCR primer pair:

(SEQ ID NO: 33)

| | |
|---|---|
| mo3Aext- | 5′-GGATCCACCATGAACTACAAGGAGTTCCTCCGC- |
| | ATGACCGCCGACAAC-3′ |

(SEQ ID NO: 34)

| | |
|---|---|
| CMS16- | 5′-CCTCCACCTGCTCCATGAAG-3′ |

The modified Cry3A085 toxin (SEQ ID NO: 11), encoded by the modified cry3A gene, has a cathepsin G recognition site, comprising the amino acid sequence AAPF (SEQ ID NO: 35), inserted in domain I between amino acids corresponding to 107 and 111 of the unmodified Cry3A toxin (SEQ ID NO: 4) and has an additional eight amino acid residues at the N-terminus of which the second residue corresponds to amino acid number 41 of the amino acid sequence set forth in SEQ ID NO: 2.

### Example 4

### Insecticidal Activity of Modified Cry3A Toxins

Modified Cry3A toxins are tested for insecticidal activity against western corn rootworm, northern corn rootworm and southern corn rootworm in insect bioassays. Bioassays are performed using a diet incorporation method. E. coli clones that express one of the modified Cry3A toxins of the invention are grown overnight. 500 μl of an overnight culture is sonicated and then mixed with 500 μl of molten artificial diet (Marrone et al. (1985) J. of Economic Entomology 78:290-293). Once the diet solidifies, it is dispensed in a petri-dish and 20 neonate corn rootworm are placed on the diet. The petri-dishes are held at 30° C. Mortality is recorded after 6 days. All of the modified Cry3A toxins cause 50%-100% mortality to western and northern corn rootworm whereas the unmodified Cry3A toxin causes 0%-30% mortality. None of the modified Cry3A toxins have activity against southern corn rootworm.

### Example 5

### Creation of Transgenic Maize Plants Comprising Modified cry3A Coding Sequences

Three modified cry3A genes, cry3A055, representative of a domain I modification, cry3A058, representative of a domain III modification, and cry3A056, representative of a domain I and domain III modification, are chosen for transformation into maize plants. An expression cassette comprising a modified cry3A coding sequence is transferred to a suitable vector for Agrobacterium-mediated maize transformation. For this example, an expression cassette comprises, in addition to the modified cry3A gene, the MTL promoter (U.S. Pat. No. 5,466,785) and the nos terminater which is known in the art. Transformation of immature maize embryos is performed essentially as described in Negrotto et al., 2000, Plant Cell Reports 19: 798-803. For this example, all media constituents are as described in Negrotto et al., supra. However, various media constituents known in the art may be substituted.

The genes used for transformation are cloned into a vector suitable for maize transformation. Vectors used in this example contain the phosphomannose isomerase (PMI) gene for selection of transgenic lines (Negrotto et al. (2000) Plant Cell Reports 19: 798-803).

Agrobacterium strain LBA4404 (pSB1) containing the plant transformation plasmid is grown on YEP (yeast extract (5 g/L), peptone (10 g/L), NaCl (5 g/L), 15 g/l agar, pH 6.8) solid medium for 2-4 days at 28° C. Approximately 0.8×10⁹ Agrobacterium are suspended in LS-inf media supplemented with 100 μM As (Negrotto et al., (2000) Plant Cell Rep 19: 798-803). Bacteria are pre-induced in this medium for 30-60 minutes. Immature embryos from A188 or other suitable genotype are excised from 8-12 day old ears into liquid LS-inf+100 μM As. Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) and cultured in the dark for 28° C. for 10 days.

Immature embryos, producing embryogenic callus are transferred to LSD1M0.5S medium. The cultures are selected on this medium for 6 weeks with a subculture step at 3 weeks. Surviving calli are transferred to Reg1 medium supplemented with mannose. Following culturing in the light (16 hour light/8 hour dark regiment), green tissues are then transferred to Reg2 medium without growth regulators and incubated for 1-2 weeks. Plantlets are transferred to Magenta GA-7 boxes (Magenta Corp, Chicago Ill.) containing Reg3 medium and grown in the light. After 2-3 weeks, plants are tested for the presence of the PMI genes and the modified cry3A genes by PCR. Positive plants from the PCR assay are transferred to the greenhouse and tested for resistance to corn rootworm.

### Example 6

### Analysis of Transgenic Maize Plants

### Corn Rootworm Efficacy

Root Excision Bioassay

Plants are sampled as they are being transplanted from Magenta GA-7 boxes into soil. This allows the roots to be sampled from a reasonably sterile environment relative to soil conditions. Sampling consists of cutting a small piece of root (ca. 2-4 cm long) and placing it onto enriched phytagar (phytagar, 12 g., sucrose, 9 g., MS salts, 3 ml., MS vitamins, 3 ml., Nystatin(25 mg/ml), 3 ml., Cefotaxime (50 mg/ml), 7 ml., Aureomycin (50 mg/ml), 7 ml., Streptomycin (50 mg/ml), 7 ml., dH₂O, 600 ml) in a small petri-dish. Negative controls are either transgenic plants that are PCR negative for the modified cry3A gene from the same experiment, or from non-transgenic plants (of a similar size to test plants) that are being grown in the phytotron. If sampling control roots from soil, the root samples are washed with water to remove soil residue, dipped in Nystatin solution (5 mg/ml), removed from the dip, blotted dry with paper toweling, and placed into a phytagar dish.

Root samples are inoculated with western corn rootworms by placing 10 first instar larvae onto the inside surface of the lid of each phytagar dish and the lids then tightly resealed. Larvae are handled using a fine tip paintbrush. After all dishes are inoculated, the tray of dishes is placed in the dark at room temperature until data collection.

At 3-4 days post inoculation, data is collected. The percent mortality of the larvae is calculated along with a visual damage rating of the root. Feeding damage is rated as high, moderate, low, or absent and given a numerical value of 3, 2, 1 or 0, respectively. Root samples causing at least 40% mortality and having a damage rating of 2 or less are considered positive.

Results in the following table show that plants expressing a modified Cry3A toxin cause from 40-100% mortality to western corn rootworm whereas control plants cause 0-30% mortality. Also, plants expressing a modified Cry3A toxin sustain significantly less feeding damage than control plants.

TABLE 2

| | | Percent Mortality | | | | | |
|---|---|---|---|---|---|---|---|
| T0 | Modified Cry3A | Per Plant | | | | | Mean Damage |
| Event | Toxin Expressed | A | B | C | D | E | Rating Per Event |
| 240A7 | Cry3A055 | 80 | 40 | 80 | 60 | | 0.8 |
| 240B2 | Cry3A055 | 60 | 60 | 60 | 80 | | 1.25 |
| 240B9 | Cry3A055 | 40 | 60 | 60 | 100 | | 1 |
| 240B10 | Cry3A055 | 80 | 40 | 60 | 60 | | 1 |
| 240A15 | Cry3A055 | 80 | 60 | 50 | 70 | 70 | 0.6 |
| 240A5 | Cry3A055 | 60 | 80 | 60 | | | 0.33 |
| 240A9 | Cry3A055 | 50 | 60 | 60 | 70 | 70 | 1.6 |
| 244A4 | Cry3A058 | 50 | | | | | 1 |
| 244A7 | Cry3A058 | 40 | 40 | 60 | | | 1.3 |
| 244A5 | Cry3A058 | 50 | | | | | 1 |
| 244B7 | Cry3A058 | 90 | | | | | 1 |
| 244B6 | Cry3A058 | 50 | 40 | 60 | | | 1 |
| 243A3 | Cry3A056 | 50 | 90 | 80 | 60 | | 1.25 |
| 243A4 | Cry3A056 | 50 | 80 | 60 | | | 1.7 |
| 243B1 | Cry3A056 | 80 | 90 | | | | 0.5 |
| 243B4 | Cry3A056 | 70 | 60 | 50 | 80 | | 1.5 |
| 245B2 | Cry3A056 | 90 | 50 | 70 | 60 | | 1 |
| WT1 | — | 0 | 10 | 20 | 10 | 0 | 2.6 |
| WT2 | — | 0 | 30 | 0 | 0 | 20 | 2.8 |

Whole Plant Bioassay

Some positive plants identified using the root excision bioassay described above are evaluated for western corn rootworm resistance using a whole plant bioassay. Plants are infested generally within 3 days after the root excision assay is completed.

Western corn rootworm eggs are preincubated so that hatch occurs 2-3 days after plant inoculation. Eggs are suspended in 0.2% agar and applied to the soil around test plants at approximately 200 eggs/plant.

Two weeks after the eggs hatch, plants are evaluated for damage caused by western corn rootworm larvae. Plant height attained, lodging, and root mass are criteria used to determine if plants are resistant to western corn rootworm feeding damage. At the time of evaluation, control plants typically are smaller than modified Cry3A plants. Also, non-transgenic control plants and plants expressing the unmodified Cry3A toxin encoded by the maize optimized cry3A gene have lodged during this time due to severe pruning of most of the roots resulting in no root mass accumulation. At the time of evaluation, plants expressing a modified Cry3A toxin of the invention are taller than control plants, have not lodged, and have a large intact root mass due to the insecticidal activity of the modified Cry3A toxin.

### ELISA Assay

ELISA analysis according to the method disclosed in U.S. Pat. No. 5,625,136 is used for the quantitative determination of the level of modified and unmodified Cry3A protein in transgenic plants.

TABLE 3
Whole Plant Bioassay Results and Protein Levels

| | | Cry3A Protein | | Intact |
|---|---|---|---|---|
| Transgenic | Type of Cry3A | Level in Roots | Plant | Root |
| Maize Plant | Toxin Expressed | (ng/mg) | Lodged | Mass |
| 240A2E | modified Cry3A055 | 224 | − | + |
| 240A9C | modified Cry3A055 | 71 | − | + |
| 240B9D | modified Cry3A055 | 204 | − | + |
| 240B9E | modified Cry3A055 | 186 | − | + |
| 240B10D | modified Cry3A055 | 104 | − | + |
| 240B10E | modified Cry3A055 | 70 | − | + |
| 240A15E | modified Cry3A055 | 122 | − | + |
| 240B4D | modified Cry3A055 | 97 | − | + |
| 243B5A | modified Cry3A056 | 41 | − | + |
| 244A7A | modified Cry3A058 | 191 | − | + |
| 710-2-51 | maize optimized | 39 | + | − |
| 710-2-54 | maize optimized | 857 | + | − |
| 710-2-61 | maize optimized | 241 | + | − |
| 710-2-67 | maize optimized | 1169 | + | − |
| 710-2-68 | maize optimized | 531 | + | − |
| 710-2-79 | maize optimized | 497 | + | − |
| 710-2-79 | maize optimized | 268 | + | − |
| WT1 Control | — | 0 | + | − |
| WT2 Control | — | 0 | + | − |

### Example 7

### Cathepsin-L Recognition Site in Modified Cry3A

Gillikin et al. (1992, Arch. Insect Biochem. 20:313-318) and others have documented that the predominant enzymes which function in the larval gut of western corn rootworm (WCR) are cysteine proteinases. One such cysteine protease has been identified as a cathepsin L (Bown et al., 2004, Insect Biochem. Mol. Biol. 34:305-320). Cathepsin L enzymes preferentially cleave peptide bonds with a hydrophobic residue in the P2 position and substrate compounds containing Phe-Arg are commonly used to assay this activity (Barrett et al., 1998, Handbook of Proteolytic Enzymes, Academic Press, New York, N.Y.). The cathepsin L proteinases from WCR readily hydrolyzed Z-Phe-Arg-AMC substrates (Bown et al., supra). These data suggest that the Cry3A055 protein (SEQ ID NO: 9) in which a cathepsin G recognition sequence (AAPF) was inserted adjacent to an arginine residue (giving AAPFR), effectively results in the introduction of both a cathepsin G recognition site and a cathepsin L recognition site.

Experiments described below support this claim as it was demonstrated that a purified cathepsin L enzyme recognized the Cry3A055 molecule (SEQ ID NO: 9) and processed it to a similar size as a chymotrypsinized-Cry3A055 product, while a unmodified Cry3A (SEQ ID NO: 4) was not processed by the cathepsin L enzyme.

Toxin preparation—E. coli-generated toxins were used for the in vitro digests and were isolated from inclusion bodies using the B-PER® Bacterial protein extraction reagent protocol (Pierce, Rockford, Ill.) per the manufacturer's instructions. Inclusion body pellets were then washed with distilled water an additional three times and solubilized in 50 mM NaHCO₃, pH 10.0 with mild shaking for 30 min at 37° C. These inclusion body preparations typically gave a single dominant band of about 67 kDa size.

In vitro processing of toxins—Sf21 cathepsin L proenzyme (R&D systems, Inc., Minneapolis, Minn.) was activated as described (Johnson and Jiang, 2005) and was buffer-exchanged into 40 mM sodium citrate, pH 3.5 plus 0.05% Tween-20 using YM-10 Microcon filters (Millipore Corp., Bedford, Mass.) for 5 cycles of concentration down to 100 μl, followed by fresh buffer addition up to 500 μl. E. coli-generated Cry3A (SEQ ID NO: 4) or Cry3A055 (SEQ ID NO: 9) protein toxins were similarly buffered-exchanged, but with YM-30 filters. All centrifugations were carried out at 14° C. in an Eppendorf 5417R microcentrifuge. In vitro processing was then examined by incubation of toxin substrates (35 ng/μl) with 11 ng/μl activated-cathepsin L in citrate buffer at room temperature. Aliquots were removed over time up to 20 h and immediately quenched with 2× Complete™ protease inhibitor cocktail (Roche Applied Science) on ice, followed by addition of Laemmli sample buffer and 100° C. incubation. Samples were separated via 12.5% Phastgel SDS-PAGE (Amersham Biosciences, Piscatawy, N.J.), transferred to nitrocellulose membrane and blocked with 2% BSA in PBS+0.05% Tween-20. Blots were then incubated with primary antibody (rabbit polyclonal anti-Cry3A) for 80 min at room temperature, washed, then incubated with secondary antibody conjugate (goat anti-rabbit-HRP, Kirkegaard & Perry Laboratories, Gaithersburg, Md.) for 1 h 45 min at room temperature. Toxin bands were then visualized using the SuperSignal® West Pico Chemiluminescence kit (Pierce, Rockford, Ill.).

Results—Cry3A055 (SEQ ID NO: 9) was susceptible to the activated Sf21 cathepsin L over time, being approximately 40% processed at the 5 h time point, and approximately 90% processed after 20 h (Table 4). In contrast, unmodified Cry3A (SEQ ID NO: 4) was not recognized by the cathepsin L, even after 20 h incubation (Table 4). A “+” denotes relative strength of the signal on a western blot. Unprocessed Cry3A (SEQ ID NO: 4) and Cry3A055 (SEQ ID NO: 9) are approximately 67 kDa proteins. A decresing intensity of the 67 kDa band with a subsequent increasing in intensity of the ˜55 kDa band demonstrates that the 67 kDa protein is being processed at the appropriate recognition site.

These results demonstrate that two functionally different non-naturally occurring protease recognition sites were introduced into Cry3A055 with the insertion of the recognition sequence AAPF (SEQ ID NO: 35). One recognition site, AAPF, is recognized by a serine protease, such as cathepsin-G, and the second recognition site, FR, is recognized by a cyteine protease, such a cathepsin-L.

TABLE 4
Results of cathepsin-L protease assays.

| Hours post-addition of | | | | | | |
|---|---|---|---|---|---|---|
| cathepsin-L enzyme for each toxin | | | | | | |
| Unmodified | | | Cry3A055 | | | |
| Cry3A (SEQ ID NO: 4) | | | (SEQ ID NO: 9) | | | Size of |
| 0 | 5 hr. | 20 hr. | 0 | 5 hr. | 20 hr. | toxin |
| ++++ | ++++ | ++++ | ++++ | ++ | + | ~67 kDa |
| + | + | + | + | ++ | ++++ | ~55 kDa |

addition up to 500 μl. E. coli-generated Cry3A (SEQ ID NO: 4) or Cry3A055 (SEQ ID NO: 9) protein toxins were similarly buffered-exchanged, but with YM-30 filters. All centrifugations were carried out at 14° C. in an Eppendorf 5417R microcentrifuge. In vitro processing was then examined by incubation of toxin substrates (35 ng/μl) with 11 ng/μl activated-cathepsin L in citrate buffer at room temperature. Aliquots were removed over time up to 20 h and immediately quenched with 2× Complete™ protease inhibitor cocktail (Roche Applied Science) on ice, followed by addition of Laemmli sample buffer and 100° C. incubation. Samples were separated via 12.5% Phastgel SDS-PAGE (Amersham Biosciences, Piscatawy, N.J.), transferred to nitrocellulose membrane and blocked with 2% BSA in PBS+0.05% Tween-20. Blots were then incubated with primary antibody (rabbit polyclonal anti-Cry3A) for 80 min at room temperature, washed, then incubated with secondary antibody conjugate (goat anti-rabbit-HRP, Kirkegaard & Perry Laboratories, Gaithersburg, Md.) for 1 h 45 min at room temperature. Toxin bands were then visualized using the SuperSignal® West Pico Chemiluminescence kit (Pierce, Rockford, Ill.).

Results—Cry3A055 (SEQ ID NO: 9) was susceptible to the activated Sf21 cathepsin L over time, being approximately 40% processed at the 5 h time point, and approximately 90% processed after 20 h (Table 4). In contrast, unmodified Cry3A (SEQ ID NO: 4) was not recognized by the cathepsin L, even after 20 h incubation (Table 4). These results demonstrate that two functionally different protease recognition sites were introduced into Cry3A055 with the insertion of the recognition sequence AAPF (SEQ ID NO: 35). One recognition site, AAPF, is recognized by a serine protease, such as cathepsin-G or chymotrypsin, and the second recognition site, FR, is recognized by a cyteine protease, such a cathepsin-L.

TABLE 4
Results of cathepsin-L enzyme assays.

| Hours post-addition of cathepsin-L | | | | | | |
|---|---|---|---|---|---|---|
| enzyme for each toxin | | | | | | Size |
| Unmodified Cry3A (SEQ ID NO: 4) | | | Cry3A055 (SEQ ID NO: 9) | | | of |
| 0 | 5 hr. | 20 hr. | 0 | 5 hr. | 20 hr. | toxin |
| ++++ | ++++ | ++++ | ++++ | ++ | + | ~67 |
| | | | | | | kDa |
| + | + | + | + | ++ | ++++ | ~55 |
| | | | | | | kDa |

What is claimed is:

1. An isolated nucleic acid molecule comprising a nucleotide sequence that encodes a modified Cry3A toxin comprising a non-naturally occurring protease recognition site, wherein said protease recognition site modifies a Cry3A toxin and is located at a position selected from the group consisting of:
a) between amino acids corresponding to amino acid numbers 107 and 115 of SEQ ID NO:4;
b) between amino acids corresponding to amino acid numbers 536 and 542 of SEQ ID NO:4; and
c) between amino acids corresponding to amino acid numbers 107 and 115 of SEQ ID NO:4, and between amino acids corresponding to amino acid numbers 536 and 542 of SEQ ID NO:4,
wherein said protease recognition site is recognizable by a gut protease of western corn rootworm, and wherein said modified Cry3A toxin causes higher mortality to western corn rootworm than the mortality caused by said Cry3A toxin to western corn rootworm in an artificial diet bioassay.

2. The isolated nucleic acid molecule according to claim 1, wherein said gut protease is a serine protease or a cysteine protease.

3. The isolated nucleic acid molecule according to claim 2, wherein said serine protease is cathepsin G.

4. The isolated nucleic acid molecule according to claim 2, wherein said cysteine protease is cathepsin L.

5. The isolated nucleic acid molecule according to claim 1, wherein said protease recognition site is located between amino acid numbers 107 and 115 of SEQ ID NO:4.

6. The isolated nucleic acid molecule according to claim 1, wherein said protease recognition site is located between amino acids corresponding to amino acid numbers 107 and 113 of SEQ ID NO:4.

7. The isolated nucleic acid molecule according to claim 6, wherein said protease recognition site is located between amino acid numbers 107 and 113 of SEQ ID NO:4.

8. The isolated nucleic acid molecule according to claim 1, wherein said protease recognition site is located between amino acids corresponding to amino acid numbers 107 and 111 of SEQ ID NO:4.

9. The isolated nucleic acid molecule according to claim 8, wherein said protease recognition site is located between amino acid numbers 107 and 111 of SEQ ID NO:4.

10. The isolated nucleic acid molecule according to claim 1, wherein said protease recognition site is located between amino acid numbers 536 and 542 of SEQ ID NO:4.

11. The isolated nucleic acid molecule according to claim 1, wherein said protease recognition site is located between amino acids corresponding to amino acid numbers 536 and 541 of SEQ ID NO:4.

12. The isolated nucleic acid molecule according to claim 11, wherein said protease recognition site is located between amino acid numbers 536 and 541 of SEQ ID NO:4.

13. The isolated nucleic acid molecule according to claim 1, wherein said protease recognition site is located between amino acids corresponding to amino acid numbers 540 and 541 of SEQ ID NO:4.

14. The isolated nucleic acid molecule according to claim 13, wherein said protease site is located between amino acid numbers 540 and 541 of SEQ ID NO:4.

15. The isolated nucleic acid molecule according to claim 1, wherein said protease recognition site is located between amino acid numbers 107 and 115 of SEQ ID NO:4 and between amino acid numbers 536 and 542 of SEQ ID NO:4.

16. The isolated nucleic acid molecule according to claim 1, wherein said protease recognition site is located between amino acids corresponding to amino acid numbers 107 and 113 of SEQ ID NO:4 and between amino acids corresponding to amino acid numbers 540 and 541 of SEQ ID NO:4.

17. The isolated nucleic acid molecule according to claim 16, wherein said protease recognition site is located between amino acid numbers 107 and 113 of SEQ ID NO:4 and between amino acid numbers 540 and 541 of SEQ ID NO:4.

18. The isolated nucleic acid molecule according to claim 1, wherein said protease recognition site is located between amino acids corresponding to amino acid numbers 107 and 111 of SEQ ID NO:4 and between amino acids corresponding to amino acid numbers 536 and 541 of SEQ ID NO:4.

19. The isolated nucleic acid molecule according to claim 18, wherein said protease recognition site is located between amino acid numbers 107 and 111 of SEQ ID NO:4 and between amino acid numbers 536 and 541 of SEQ ID NO:4.

20. The isolated nucleic acid molecule according to claim 1, wherein said protease recognition site is located between amino acids corresponding to amino acid numbers 107 and 111 of SEQ ID NO:4 and between amino acids corresponding to amino acid numbers 540 and 541 of SEQ ID NO:4.

21. The isolated nucleic acid molecule according to claim 20, wherein said protease recognition site is located between amino acid numbers 107 and 111 of SEQ ID NO:4 and between amino acid numbers 540 and 541 of SEQ ID NO:4.

22. The isolated nucleic acid molecule according to claim 1, wherein said modified Cry3A toxin causes at least 50% mortality to western corn rootworm to which said Cry3A toxin causes up to 30% mortality.

23. The isolated nucleic acid molecule according to claim 1, wherein said nucleotide sequence comprises SEQ ID NO: 6, SEQ ID NO: 8, SEQ ID NO: 10, SEQ ID NO: 12, SEQ ID NO: 14, SEQ ID NO: 16, SEQ ID NO: 18, or SEQ ID NO: 20.

24. The isolated nucleic acid molecule according to claim 1, wherein said modified Cry3A toxin comprises the amino acid sequence set forth in SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 21.

25. The isolated nucleic acid molecule according to claim 1, wherein said modified Cry3A toxin is active against northern corn rootworm.

26. A chimeric construct comprising a heterologous promoter sequence operatively linked to the nucleic acid molecule of claim 1.

27. A recombinant vector comprising the chimeric construct of claim 26.

28. A transgenic non-human host cell comprising the chimeric construct of claim 26.

29. The transgenic host cell according to claim 28, which is a bacterial cell.

30. The transgenic host cell according to claim 28, which is a plant cell.

31. A transgenic plant comprising the transgenic plant cell of claim 30.

32. The transgenic plant according to claim 31, wherein said plant is a maize plant.

33. Transgenic seed from the transgenic plant of claim 31, wherein said seed comprises the nucleic acid molecule.

34. Transgenic seed from the maize plant of claim 32, wherein said seed comprises the nucleic acid molecule.

35. An isolated toxin produced by the expression of the nucleic acid molecule according to claim 1.

36. The transgenic maize plant according to claim 32, wherein said nucleotide sequence comprises SEQ ID NO: 8, SEQ ID NO: 14 or SEQ ID NO: 18.

37. The transgenic maize plant according to claim 32, wherein said modified Cry3A toxin comprises SEQ ID NO: 9, SEQ ID NO: 15 or SEQ ID NO: 19.

38. The transgenic maize plant according to claim 32, wherein said root tissue causes 100% mortality to western corn rootworm.

39. The transgenic maize plant according to claim 32, wherein said root tissue causes 90% mortality to western corn rootworm.

40. The transgenic maize plant according to claim 32, wherein said root tissue causes 80% mortality to western corn rootworm.

41. The transgenic maize plant according to claim 32, wherein said root tissue causes 70% mortality to western corn rootworm.

42. The transgenic maize plant according to claim 32, wherein said root tissue causes 60% mortality to western corn rootworm.

43. The transgenic maize plant according to claim 32, wherein said root tissue causes 50% mortality to western corn rootworm.

44. The transgenic maize plant according to claim 32, wherein said root tissue causes 40% mortality to western corn rootworm.

45. The transgenic maize plant according to claim 32, wherein said transgenic plant expresses said modified Cry3A toxin at a level sufficient to prevent western corn rootworm from severely pruning the roots of the transgenic plant.

46. The transgenic maize plant according to claim 32, wherein said transgenic plant expresses said modified Cry3A toxin at a level sufficient to prevent western corn rootworm feeding damage from causing the plant to lodge.

47. The transgenic maize plant according to claim 32, which is an inbred plant.

48. The transgenic maize plant according to claim 32, which is a hybrid plant.

49. Transgenic seed from the plant of claim 47, wherein said seed comprises the nucleic acid molecule.

50. Transgenic seed from the plant of claim 48, wherein said seed comprises the nucleic acid molecule.

51. A modified Cry3A toxin comprising a non-naturally occurring protease recognition site, wherein said protease recognition site modifies a Cry3A toxin and is located at a position selected from the group consisting of:
a) between amino acids corresponding to amino acid numbers 107 and 115 of SEQ ID NO:4;
b) between amino acids corresponding to amino acid numbers 536 and 542 of SEQ ID NO:4; and
c) between amino acids corresponding to amino acid numbers 107 and 115 of SEQ ID NO:4, and between amino acids corresponding to amino acid numbers 536 and 542 of SEQ ID NO:4,
wherein said protease recognition site is recognizable by a gut protease of western corn rootworm, and wherein said modified Cry3A toxin causes higher mortality to western corn rootworm than the mortality caused by said Cry3A toxin to western corn rootworm in an artificial diet bioassay.

52. The modified Cry3A toxin according to claim 51, wherein said gut protease is a serine protease or a cysteine protease.

53. The modified Cry3A toxin according to claim 52, wherein said serine protease is cathepsin G.

54. The modified Cry3A toxin according to claim 52, wherein said cysteine protease is cathepsin L.

55. The modified Cry3A toxin according to claim 51, wherein said protease recognition site is located between amino acid numbers 107 and 115 of SEQ ID NO:4.

56. The modified Cry3A toxin according to claim 51, wherein said protease recognition site is located between amino acids corresponding to amino acid numbers 107 and 113 of SEQ ID NO:4.

57. The modified Cry3A toxin according to claim 56, wherein said protease recognition site is located between amino acid numbers 107 and 113 of SEQ ID NO:4.

58. The modified Cry3A toxin according to claim 51, wherein said protease recognition site is located between amino acids corresponding to amino acid numbers 107 and 111 of SEQ ID NO:4.

59. The modified Cry3A toxin according to claim 58, wherein said protease recognition site is located between amino acid numbers 107 and 111 of SEQ ID NO:4.

60. The modified Cry3A toxin according to claim 51, wherein said protease site is located between amino acid numbers 536 and 542 of SEQ ID NO:4.

61. The modified Cry3A toxin according to claim 51, wherein said protease recognition site is located between amino acids corresponding to amino acid numbers 536 and 541 of SEQ ID NO:4.

62. The modified Cry3A toxin according to claim 61, wherein said protease recognition site is located between amino acid numbers 536 and 541 of SEQ ID NO:4.

63. The modified Cry3A toxin according to claim 51, wherein said protease recognition site is located between amino acids corresponding to amino acid numbers 540 and 541 of SEQ ID NO:4.

64. The modified Cry3A toxin according to claim 63, wherein said protease recognition site is located between amino acid numbers 540 and 541 of SEQ ID NO:4.

65. The modified Cry3A toxin according to claim 51, wherein said protease recognition site is located between amino acid numbers 107 and 115 and between amino acid numbers 536 and 542 of SEQ ID NO:4.

66. The modified Cry3A toxin according to claim 51, wherein said protease recognition site is located between amino acids corresponding to amino acid numbers 107 and 113 of SEQ ID NO:4 and between amino acids corresponding to amino acid numbers 540 and 541 of SEQ ID NO:4.

67. The modified Cry3A toxin according to claim 66, wherein said protease recognition site is located between amino acid numbers 107 and 113 of SEQ ID NO:4 and between amino acid numbers 541 and 541 of SEQ ID NO:4.

68. The modified Cry3A toxin according to claim 51, wherein said protease recognition site is located between amino acids corresponding to amino acid numbers 107 and 111 of SEQ ID NO:4 and between amino acids corresponding to amino acid numbers 536 and 541 of SEQ ID NO:4.

69. The modified Cry3A toxin according to claim 68, wherein said protease recognition site is located between amino acid numbers 107 and 111 of SEQ ID NO:4 and between amino acid numbers 536 and 541 of SEQ ID NO:4.

70. The modified Cry3A toxin according to claim 51, wherein said protease recognition site is located between amino acids corresponding to amino acid numbers 107 and 111 of SEQ ID NO:4 and between amino acids corresponding to amino acid numbers 540 and 541 of SEQ ID NO:4.

71. The modified Cry3A toxin according to claim 70, wherein said protease recognition site is located between amino acid numbers 107 and 111 of SEQ ID NO:4 and between amino acid numbers 540 and 541 of SEQ ID NO:4.

72. The modified Cry3A toxin according to claim 51, wherein said modified Cry3A toxin causes at least 50% mortality to western corn rootworm to which said Cry3A toxin causes up to 30% mortality.

73. The modified Cry3A toxin according to claim 51, wherein said toxin is encoded by SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

74. The modified Cry3A toxin according to claim 51, wherein sadi toxin comprises SEQ ID NO: 7, SEQ ID NO: 9, SEQ ID NO: 11, SEQ ID NO: 13, SEQ ID NO: 15, SEQ ID NO: 17, SEQ ID NO: 19 or SEQ ID NO: 21.

75. The modified Cry3A toxin according to claim 51 which is active against northern corn rootworm.

76. A composition comprising an effective amount of the modified Cry3A toxin of claim 51 to cause mortality to western corn rootworm.

77. A method of controlling infestation of maize plants by western corn rootworm, the method comprising:
(a) providing the transgenic maize plant according to claim 32; and
(b) contacting said western corn rootworm with the plant.

78. A method of producing a modified Cry3A, comprising:
(a) obtaining the transgenic host cell according to claim 28;
(b) culturing the transgenic host cell under conditions that allow the expression of the modified Cry3A toxin; and
(c) recovering the expressed modified Cry3A toxin.

79. A method of producing insect-resistant plants, comprising:
(a) stably integrating the nucleic acid molecule according to claim 1 into the genome of plant cells; and
(b) regenerating stably transformed plants from said transformed plant cells, wherein said stably transformed plants express an effective amount of a modified Cry3A
toxin to render said transformed plant resistant to at least western corn rootworm.

80. A method of controlling at least western corn rootworm, comprising delivering orally to western corn rootworm an effective amount of a toxin according to claim 51.

81. A method of making a modified Cry3A toxin, comprising:
(a) obtaining a cry3A gene which encodes a Cry3A toxin;
(b) obtaining a nucleotide sequence which encodes a protease recognition;
(c) inserting said nucleotide sequence into said cry3A gene, such that said protease recognition site is located in said Cry3A toxin at a position between amino acids corresponding to amino acid numbers 107 and 115 of SEQ ID NO:4, at a position between amino acids corresponding to amino acid numbers 536 and 542 of SEQ ID NO:4, or at a position between amino acids corresponding to amino acid numbers 107 and 115 of SEQ ID NO:4 and between amino acids corresponding to amino acid numbers 536 and 542 of SEQ ID NO:4, thus creating a modified cry3A gene;
(d) inserting said modified cry3A gene into an expression cassette; and
(e) transforming said expression cassette into a non-human host cell, wherein said host cell produces a modified Cry3A toxin.

82. A modified cry3A gene comprising a nucleotide sequence that encodes a modified Cry3A toxin comprising a non-naturally occurring protease recognition site, wherein said modified cry3A gene comprises a coding sequence encoding said protease recognition site, wherein said coding sequence modifies a cry3A gene and is inserted at a position selected from the group consisting of:
a) between the codons that code for amino acids corresponding to amino acid numbers 107 and 115 of SEQ ID NO:4;
b) between the codons that code for amino acids corresponding to amino acid numbers 536 and 542 of SEQ ID NO:4; and
c) between the codons that code for amino acids corresponding to amino acid numbers 107 and 115 of SEQ ID NO:4, and between codons that code for amino acids corresponding to amino acid numbers 536 and 542 of SEQ ID NO:4,
wherein said protease recognition site is recognizable by a gut protease of western corn rootworm, and wherein said modified Cry3A toxin causes higher mortality to western corn rootworm than the mortality caused by said Cry3A toxin to western corn rootworm in an artificial diet bioassay.

83. The modified cry3A gene according to claim 82, wherein said gut protease is a serine protease or a cysteine protease.

84. The modified cry3A gene according to claim 83, wherein said serine protease is cathepsin G.

85. The modified cry3A gene according to claim 83, wherein said cysteine protease is cathepsin L.

86. The modified cry3A gene according to claim 82, wherein said nucleotide sequence comprises SEQ ID NO: 6, SEQ ID NO: 8, SEQ ID NO: 10, SEQ ID NO: 12, SEQ ID NO: 14, SEQ ID NO: 16, SEQ ID NO: 18, or SEQ ID NO: 20.

87. The modified cry3A gene according to claim 82, wherein said modified Cry3A toxin comprises SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 21.

88. The modified cry3A gene according to claim 82, wherein said modified Cry3A toxin is active against northern corn rootworm.

89. A chimeric construct comprising a heterologous promoter sequence operatively linked to the modified cry3A gene of claim 82.

90. A recombinant vector comprising the chimeric construct of claim 89.

91. A transgenic non-human host cell comprising the chimeric construct of claim 89.

92. The transgenic host cell according to claim 91, which is a bacterial cell.

93. The transgenic host cell according to claim 91, which is a plant cell.

94. A transgenic plant comprising the transgenic plant cell of claim 93.

95. The transgenic plant according to claim 94, wherein said plant is a maize plant.

96. Transgenic seed from the transgenic plant of claim 94, wherein said seed comprises the modified cry3A gene.

97. Transgenic seed from the maize plant of claim 95, wherein said seed comprises the modified cry3A gene.

98. The transgenic maize plant according to claim 95, wherein said nucleotide sequence comprises SEQ ID NO: 8, SEQ ID NO: 14, or SEQ ID NO: 18.

99. The transgenic maize plant according to claim 95, wherein said modified Cry3A toxin comprises SEQ ID NO: 9, SEQ ID NO: 15, or SEQ ID NO: 19.

100. The transgenic maize plant according to claim 95, which is an inbred plant.

101. The transgenic maize plant according to claim 95, which is a hybrid plant.

102. Transgenic seed from the plant of claim 100, wherein said seed comprises the modified cry3A gene.

103. Transgenic seed from the plant of claim 101, wherein said seed comprises the modified cry3A gene.
